(19) United States
(12) Patent Application Publication
(10) Pub. No.: US 2021/0401440 A1
(43) Pub. Date: Dec. 30, 2021

(54) Janjua Aneurysm Net with a Stiff Proximal Portion and a Flexible Distal Portion

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 17/472,674

(22) Filed: Sep. 12, 2021

Related U.S. Application Data
(63) Continuation-in-part of application No. 17/467,680, filed on Sep. 7, 2021.
Application No. 17/467,680 is a continuation-in-part of application No. 17/466,497, filed on Sep. 3, 2021.
Application No. 17/466,497 is a continuation-in-part of application No. 17/353,652, filed on Jun. 21, 2021.
Application No. 17/353,652 is a continuation-in-part of application No. 17/220,002, filed on Apr. 1, 2021.
Application No. 17/220,002 is a continuation-in-part of application No. 17/214,827, filed on Mar. 27, 2021.
Application No. 17/214,827 is a continuation-in-part of application No. 17/211,446, filed on Mar. 24, 2021.
Application No. 17/211,446 is a continuation-in-part of application No. 16/693,267, filed on Nov. 23, 2019.
Application No. 16/693,267 is a continuation-in-part of application No. 16/660,929, filed on Oct. 23, 2019.
Application No. 16/660,929 is a continuation-in-part of application No. 16/541,241, filed on Aug. 15, 2019.
Application No. 17/220,002 is a continuation-in-part of application No. 17/214,827, filed on Mar. 27, 2021.
Application No. 17/214,827 is a continuation-in-part of application No. 17/211,446, filed on Mar. 24, 2021.
Application No. 17/211,446 is a continuation-in-part of application No. 16/693,267, filed on Nov. 23, 2019.
Application No. 16/693,267 is a continuation-in-part of application No. 16/660,929, filed on Oct. 23, 2019.
Application No. 16/693,267 is a continuation-in-part of application No. 16/660,929, filed on Oct. 23, 2019.
Application No. 16/660,929 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12113; A61B 17/12177

(57) ABSTRACT

This invention is an intrasacular aneurysm occlusion device comprising a flexible net or mesh with a proximal portion and a distal portion which is inserted into and expanded within an aneurysm sac. The proximal portion is stiffer and/or less flexible than the distal portion. The device also includes an opening through which embolic members and/or embolic material is inserted into the flexible net or mesh. Insertion of embolic members and/or material causes the flexible net or mesh to expand and conform to the walls of even an irregularly-shaped aneurysm sac.

### CROSS-REFERENCE TO RELATED APPLICATIONS

This application is a continuation-in-part of U.S. patent application Ser. No. 17/467,680 filed on 2021 Sep. 7. This application is a continuation-in-part of U.S. patent application Ser. No. 17/466,497 filed on 2021 Sep. 3. This application is a continuation-in-part of U.S. patent application Ser. No. 17/353,652 filed on 2021 Jun. 21. This application is a continuation-in-part of U.S. patent application Ser. No. 17/220,002 filed on 2021 Apr. 1. This application is a continuation-in-part of U.S. patent application Ser. No. 17/214,827 filed on 2021 Mar. 27. This application is a continuation-in-part of U.S. patent application Ser. No. 17/211,446 filed on 2021 Mar. 24. This application claims the priority benefit of U.S. provisional patent application 63/119,774 filed on 2020 Dec. 1. This application is a continuation-in-part of U.S. patent application Ser. No. 16/693,267 filed on 2019 Nov. 23. This application is a continuation-in-part of U.S. patent application Ser. No. 16/660,929 filed on 2019 Oct. 23. This application is a continuation-in-part of U.S. patent application Ser. No. 16/541,241 filed on 2019 Aug. 15.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application 17/220,002 was a continuation-in-part of U.S. patent application Ser. No. 16/660929 filed on 2019 Oct. 23.

U.S. patent application 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application 16/541,241 filed on 2019 Aug. 15. U.S. patent application Ser. No. 16,693,267 was a continuation-in-part of U.S. patent application Ser. No. 15/865,822 filed on 2018 Jan. 9 which issued as U.S. Pat. No. 10,716,573 on 2020 Jul. 21 U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application 15/865,822 filed on 2018 Jan. 9 which issued as U.S. Pat. No. 10,716,573 on 2020 Jul. 21 U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21

U.S. patent application 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application 15081909 filed on 2016-03-27. U.S. patent application Ser. No. 15/865,822 was a continuation-in-part of U.S. patent application Ser. No. 14/526,600 filed on 2014 Oct. 29.

U.S. patent application Ser. No. Ser. No. 15/861,482 claimed the priority benefit of U.S. provisional patent application 62/589,754 filed on 2017 Nov. 22. U.S. patent application 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application 15/861,482 was a continuation-in-part of U.S. patent application 15/080,915 filed on 2016 Mar. 25 which issued as U.S. Pat. No. 10,028,747 on 2018 Jul. 24 U.S. patent application Ser. No. 15/861,482 was a continuation-in-part of U.S. patent application Ser. No. 14/526,600 filed on 2014 Oct. 29.

U.S. patent application 15/081,909 was a continuation-in-part of U.S. patent application Ser. No. 14/526,600 filed on 2014 Oct. 29. U.S. patent application 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10 U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61,126,027 filed on 2008 May 1.

The entire contents of these related applications are incorporated herein by reference.

### FEDERALLY SPONSORED RESEARCH: Not Applicable

### SEQUENCE LISTING OR PROGRAM: Not Applicable

### BACKGROUND

### Field of Invention

This invention relates to devices for occluding cerebral aneurysms.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE RELEVANT ART

U.S. Pat. No. 8,998,947 (Aboytes et al., Apr. 7, 2015, “Devices and Methods for the Treatment of Vascular Defects”) discloses an expandable implant with a plurality of flattened, petal-shaped portions. U.S. patent application 20210169496 (Badruddin et al., Jun. 10, 2021, “System for and Method of Treating Aneurysms”) discloses an apparatus with a wire to be advanced within a tube and an occlusion element disposed on the wire, a cover, and an inner anchoring member. U.S. patent applications 20170079661 (Bardsley et al., Mar. 23, 2017, “Occlusive Devices”) and 20190269411 (Bardsley et al., Sep. 5, 2019, “Occlusive Devices”) and U.S. Pat. No. 10,314,593 (Bardsley et al., Jun. 11, 2019, “Occlusive Devices”) disclose an implant with a single-layer or dual-layer braided body having a variable porosity.

U.S. Pat. No. 9,585,669 (Becking et al., Mar. 17, 2017, “Multiple Layer Filamentary Devices for Treatment of Vascular Defects”) discloses a self-expanding resilient permeable shell with a proximal end, a distal end, a longitudinal axis, and a plurality of elongate resilient filaments. U.S. Pat. No. 10,980,545 (Bowman et al., Apr. 20, 2021, “Devices for Vascular Occlusion”) discloses a braided wire device with a linear compressed shape within a catheter and an expanded state that expands away from an axis of a distal end a delivery pusher in a longitudinally angled and an axially offset manner. U.S. patent application 20210228214 (Bowman et al., Jul. 29, 2021, “Devices for Vascular Occlusion”) discloses a mesh neck bridge with an opening.

U.S. patent application 20110022149 (Cox et al., Jan. 27, 2011, “Methods and Devices for Treatment pf Vascular Defects”) discloses an expandable body support structure with first ends secured to a first ring and second ends secured to a second ring. U.S. patent application 20120165919 (Cox et al., Jun. 28, 2012, “Methods and Devices for Treatment of Vascular Defects”) discloses an expandable wire body support structure with a substantially spherical or globular configuration and a portion with low or no porosity. U.S. patent application 20120283768 (Cox et al., Nov. 8, 2012, “Method and Apparatus for the Treatment of Large and Giant Vascular Defects”) discloses deployment of multiple permeable shell devices. U.S. patent application 20140052233 (Cox et al., Feb. 20, 2014, “Methods and Devices for Treatment of Vascular Defects”) discloses a method for treating a cerebral aneurysm by expanding a substantially spherical or globular shell.

U.S. provisional patent application 62/819,317 (Dholakia et al., Mar. 15, 2019, “Occlusion”) discloses intrasaccular occlusive devices that utilize an apple-core braid winding shape. U.S. patent application 20200289125 (Dholakia et al., Sep. 17, 2020, “Filamentary Devices Having a Flexible Joint for Treatment of Vascular Defects”) discloses an implant with first and second permeable shells. U.S. patent applications 20140135812(Divino et al., May 15, 2014, “Occlusive Devices”), 20190282242 (Divino et al., Sep. 19, 2019, “Occlusive Devices”), 20190290286 (Divino et al., Sep. 26, 2019, “Occlusive Devices”) and 20190343532 (Divino et al., Nov. 14, 2019, “Occlusive Devices”) and U.S. Pat. No. 10,327,781 (Divino et al., Jun. 25, 2019, “Occlusive Devices”) disclose a device with at least one expandable structure adapted to transition from a compressed configuration to an expanded configuration when released into the aneurysm.

U.S. patent application 20200155333 (Franano et al., May 21, 2020, “Ballstent Device and Methods of Use”) discloses a rounded, thin-walled, expandable metal structure (“ballstent”). U.S. Pat. No. 11,013,516 (Franano et al., May 25, 2021, “Expandable Body Device and Method of Use”) discloses a single-lobed, thin-walled, expandable body (“ballstent” or “blockstent”) and a flexible, elongated delivery device (“delivery catheter”). U.S. Pat. No. 11,033,275 (Franano et al., Jun. 15, 2021, “Expandable Body Device and Method of Use”) discloses hollow gold structures that can be folded, wrapped, compressed, advanced to a location in the body of patient, and expanded by injection of a fluid.

U.S. patent application 20210085333 (Gorochow et al., Mar. 25, 2021, “Inverting Braided Aneurysm Treatment System and Method”) discloses a tubular braid with an open end, a pinched end, and a predetermined shape. U.S. patent application 20210145449 (Gorochow, May 20, 2021, “Implant Delivery System with Braid Cup Formation”) discloses an implant system with an engagement wire, a pull wire, and a braided implant having a distal ring thereon. U.S. patent application 20210169495 (Gorochow et al., Jun. 10, 2021, “Intrasaccular Inverting Braid with Highly Flexible Fill Material”) discloses a tubular braided implant including a braid that can be delivered as a single layer braid, invert into itself during deployment to form at least two nested sacks and an additional braid material that can fill the innermost sack.

U.S. patent application 20210169498 (Gorochow, Jun. 10, 2021, “Delivery of Embolic Braid”) discloses a method for a braided implant with a band attached to a delivery tube. U.S. patent application 20210186518 (Gorochow et al., Jun. 24, 2021, “Implant Having an Intrasaccular Section and Intravascular Section”) discloses a tubular braid with an intrasaccular section, an intravascular section, a pinched section, and a predetermined shape. U.S. patent application 20210196284 (Gorochow et al., Jul. 1, 2021, “Folded Aneurysm Treatment Device and Delivery Method”) and U.S. Pat. No. 11,076,861 (Gorochow et al., Aug. 3, 2021, “Folded Aneurysm Treatment Device and Delivery Method”) disclose a device with a braided implant within an aneurysm sack such that an outer non-inverted layer contacts a wall of the aneurysm and an inverted layer apposes the outer non-inverted layer to form a double layer of braid across a neck of the aneurysm. U.S. Pat. No. 11,051,825 (Gorochow, Jul. 6, 2021, “Delivery System for Embolic Braid”) discloses a braided implant attached to a releasing component that can be detachably engaged with a delivery tube and a pull wire. U.S. Pat. No. 11,058,430 (Gorochow et al., Jul. 13, 2021, “Aneurysm Device and Delivery System”) discloses a braided device with a proximal expandable portion for sealing an aneurysm neck and a distal expandable portion.

U.S. patent application 20190216467 (Goyal, Jul. 18, 2019, “Apparatus and Methods for Intravascular Treatment of Aneurysms”) discloses a device with a first portion having an expandable and compressible mesh for expansion against the wall of an aneurysm and a second disk portion covering an outside of the neck opening. U.S. patent application 20180070955 (Greene et al., Mar. 15, 2018, “Embolic Containment”) discloses a method of treating a neurovascular arteriovenous malformation comprising a catheter with a mesh catch structure on the distal portion of the catheter, wherein the catheter is configured to deliver liquid embolic and dimethyl sulfoxide.

U.S. patent application 20190059909 (Griffin, Feb. 28, 2019, “Occlusion Device”) discloses an occlusion device with a marker and a low profile resilient mesh body attached to the distal end of the marker, the body having a delivery shape and a deployed shape capable of conforming to aneurysm walls. U.S. Pat. No. 10,285,711 (Griffin, May 14, 2019, “Occlusion Device”) discloses a continuous compressible mesh structure comprising axial mesh carriages configured end to end, wherein each end of each carriage is a pinch point in the continuous mesh structure. U.S. patent application 20210068842 (Griffin, Mar. 11, 2021, “Occlusion Device”) discloses an occlusion device with a marker band and a resilient mesh body attached within the marker band. U.S. patent application 20210153871 (Griffin, May 27, 2021, “Occlusion Device”) discloses a continuous mesh structure comprising a medial pinch point.

U.S. provisional patent application 61/866,993 (Hewitt et al., Aug, 16, 2013, “Filamentary Devices for Treatment of Vascular Defects”) discloses a self-expanding resilient permeable structure wherein at least some elongate filaments include highly radiopaque material. U.S. provisional patent application 61/979,416 (Hewitt et al, Apr. 14, 2014, “Devices for Therapeutic Vascular Procedures”) discloses a self-expanding resilient permeable shell with a plurality of elongate resilient filaments having a variable braided structure. U.S. patent application 20140358178 (Hewitt et al., Dec. 4, 2014, “Filamentary Devices for Treatment of Vascular Defects”) discloses a resilient self-expanding permeable shell with at least 40% composite filaments relative to a total number of filaments, wherein composite filaments comprise a high strength material and a highly radiopaque material. U.S. provisional patent application 62/093,313 (Hewitt et al., Dec. 17, 2014, “Devices for Therapeutic Vascular Procedures”) discloses a self-expanding resilient permeable shell with elongate resilient filaments having a variable braided structure, wherein a distal portion has a first braid density, a proximal portion has a second braid density, and the second braid density is greater than the first braid density.

U.S. patent application 20160249935 (Hewitt et al., Sep. 1, 2016, “Devices for Therapeutic Vascular Procedures”) discloses an expandable cylindrical structure made of wires and a self-expanding permeable shell at the distal end of the cylindrical structure. U.S. patent application 20160249934 (Hewitt et al., Sep. 1, 2016, “Filamentary Devices for Treatment of Vascular Defects”) discloses a woven braided mesh having variable mesh density. U.S. patent application 20160367260 (Hewitt et al., Dec. 22, 2016, “Devices for Therapeutic Vascular Procedures”) and U.S. Pat. No. 9,629,635 (Hewitt et al., Apr. 25, 2017, “Devices for Therapeutic Vascular Procedures”) disclose an expandable structure with distal and proximal permeable shells having different pore sizes. U.S. patent application 20170128077 (Hewitt et al., May 11, 2017, “Devices for Therapeutic Vascular Procedures”) discloses a self-expanding resilient permeable shell with a metallic coil secured at a distal end.

U.S. patent applications 20180206849 (Hewitt et al., Jul. 26, 2018, “Filamentary Devices for the Treatment of Vascular Defects”) and 20200289126 (Hewitt et al., Sep. 17, 2020, “Filamentary Devices for Treatment of Vascular Defects”) and U.S. Pat. No. 9,955,976 (Hewitt et al., May 1, 2018, “Filamentary Devices for Treatment of Vascular Defects”) and U.S. Pat. No. 10,939,914 (Hewitt et al., Mar. 9, 2021, “Filamentary Devices for the Treatment of Vascular Defects”) disclose mesh balls with different layers and areas with different porosities. U.S. patent application 20190223881 (Hewitt et al., Jul. 25, 2019, “Devices for Therapeutic Vascular Procedures”) discloses a self-expanding resilient permeable shell whose filaments have a distal region that extends beyond the distal end of the permeable shell and forms an extension having a generally-circular shape. U.S. patent application 20210106337 (Hewitt et al., Apr. 15, 2021, “Filamentary Devices for Treatment of Vascular Defects”) discloses a resilient self-expanding permeable implant with an expanded state with a longitudinally shortened configuration.

U.S. provisional patent application 61/483,032 (Kent et al., May 5, 2011, “Method and Apparatus for the Treatment of Large and Giant Vascular Defects”) discloses various self-expanding shells, including some with double shells and layers. U.S. patent application 20210128169 (Li et al., May 6, 2021, “Devices, Systems, and Methods for Treatment of Intracranial Aneurysms”) discloses systems and methods for treating an aneurysm including intravascularly delivering an occlusive member to an aneurysm cavity and deforming a shape of the occlusive member via introduction of an embolic element to a space between the occlusive member and an inner surface of the aneurysm wall.

U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) disclose a device with a control ring having a substantially annular body disposed on the proximal end region to prevent radial expansion of the proximal end region and to provide an engagement feature during manipulation of the occlusion device. U.S. patent application 20210007755 (Lorenzo et al., Jan. 14, 2021, “Intrasaccular Aneurysm Treatment Device With Varying Coatings”) discloses an implant with a braided mesh movable from a delivery configuration having a single-layer tubular shape to an implanted configuration sized to be implanted in an aneurysm sac. U.S. Pat. No. 10,716,574 (Lorenzo et al., Jul. 21, 2020, “Aneurysm Device and Delivery Method”) discloses a self-expanding braided device with an inverted outer occlusive sack.

U.S. patent application 20200375606 (Lorenzo, Dec. 3, 2020, “Aneurysm Method and System”) discloses a self-expanding braided implant with a distal implant end and a proximal implant end, the braided implant being invertible about the distal implant end. U.S. Pat. No. 10,905,430 (Lorenzo et al., Feb. 2, 2021, “Aneurysm Device and Delivery System”) discloses a braided device with inner and outer meshes. U.S. patent application 20210177429 (Lorenzo, Jun. 17, 2021, “Aneurysm Method and System”) discloses a vaso-occlusive device with at least two nested sacks. U.S. Pat. No. 11,076,860 (Lorenzo, Aug. 3, 2021, “Aneurysm Occlusion Device”) discloses a tubular structure having a proximal end region and a distal end region, having an expanded condition and a collapsed condition.

U.S. patent application 20130245667 (Marchand et al., Sep. 19, 2013, “Filamentary Devices and Treatment of Vascular Defects”) discloses a self-expanding resilient permeable shell with filaments which are bundled and secured to each other at a proximal end. U.S. patent application 20160249937 (Marchand et al., Sep. 1, 2016, “Multiple Layer Filamentary Devices for Treatment of Vascular Defects”) discloses an occlusion device with a number of undulations. U.S. patent application 20180000489 (Marchand et al., Jan. 4, 2018, “Filamentary Devices for Treatment of Vascular Defects”) and U.S. Pat. No. 10,610,231 (Marchand et al., Apr. 7, 2020, “Filamentary Devices for Treatment of Vascular Defects”) disclose a self-expanding resilient permeable shell wherein a ratio of the total cross-sectional area of small filaments to the total cross-sectional area of large filaments is between 0.56 and 1.89. U.S. patent application 20200281603 (Marchand et al., Sep. 10, 2020, “Filamentary Devices for Treatment of Vascular Defects”) discloses a permeable shell with a swellable polymer.

U.S. patent applications 20180271540 (Merritt et al., Sep. 27, 2018, “Systems and Methods for Embolization of Body Structures”) and 20210169499 (Merritt et al., Jun. 10, 2021, “Systems and Methods for Embolization of Body Structures”) disclose a self-expanding permeable shell with a plurality of circumferentially-arrayed lobes. U.S. patent application 20210007754 (Milhous et al., Jan. 14, 2021, “Filamentary Devices for Treatment of Vascular Defects”) discloses inner and outer mesh balls. U.S. provisional patent application 62/873,256 (Milhous et al., Jul. 12, 2019, “Devices for Treatment of Vascular Defects”) discloses a mesh of braided wires gathered into retention structures at proximal and distal ends. U.S. patent application 20210129275 (Nguyen et al., May 6, 2021, “Devices, Systems, and Methods for Treating Aneurysms”) discloses a method of everting a mesh such that the mesh encloses an open volume with a shape based, at least in part, on the shape of a forming assembly.

U.S. patent application 20210128168 (Nguyen et al., May 6, 2021, “Systems and Methods for Treating Aneurysms”) discloses a treatment system with an electrolytically corrodible conduit having a proximal portion, a distal portion, and a detachment zone between the proximal portion and the distal portion. U.S. patent applications 20210128167 (Patel et al., May 6, 2021, “Systems and Methods for Treating Aneurysms”) and 20210128160 (Li et al., May 6, 2021, “Systems and Methods for Treating Aneurysms”) disclose the use of an occlusive member (e.g., an expandable braid) in conjunction with an embolic element (e.g., coils, embolic material). U.S. Pat. No. 11,058,431 (Pereira et al., Jul. 13, 2021, “Systems and Methods for Treating Aneurysms”) discloses an occlusion element having a distal end, a proximal end, and a longitudinal axis extending between the distal end and the proximal end, the occlusion element configured to be delivered in a collapsed configuration and further configured to expand to an expanded configuration, and the occlusion element comprising an inverted mesh tube having an outer layer and an inner layer.

U.S. provisional patent application 62/307,123 (Plaza et al, Mar. 11, 2016, “Systems and Methods for Delivery of Stents and Stent-like Devices”) appears to disclose an expanding aneurysm occlusion device which is implantable within the parent vessel of an aneurysm. U.S. patent application 20170258473 (Plaza et al., Sep. 14, 2017, “Systems and Methods for Delivery of Stents and Stent-Like Devices”) and U.S. Pat. No. 10,952,739 (Plaza et al., Mar. 23, 2021, “Systems and Methods for Delivery of Stents and Stent-Like Devices”) disclose an expandable elongate tubular member. U.S. patent application 20210052279 (Porter et al., Feb. 25, 2021, “Intra-Aneurysm Devices”) discloses a device with an upper member that sits against the dome of an aneurysm, a lower member that sits in the neck of the aneurysm, and a means of adjusting the overall dimensions of the device.

U.S. patent application 20180092690 (Priya et al., Apr. 5, 2018, “Customized Endovascular Devices and Methods Pertaining Thereto”) discloses patient-specific 3D complex coils and methods of making such coils, including custom fixtures for the manufacture of such coils. U.S. patent application 20210128165(Pulugurtha et al., May 6, 2021, “Systems and Methods for Treating Aneurysms”) discloses an occlusive member configured to be positioned within an aneurysm sac, and a distal conduit coupled to the occlusive member and having a first lumen extending therethrough.

U.S. provisional patent application 62/819,296 (Rangwala et al, Mar. 15, 2019, “Occlusion”) discloses an intrasaccular occlusive device with a more flexible distal section and a more stiff proximal section. U.S. patent application 20200289124 (Rangwala et al., Sep. 17, 2020, “Filamentary Devices for Treatment of Vascular Defects”) discloses a permeable implant with a stiffer proximal portion near the neck of an aneurysm. U.S. patent applications 20210128162 (Rhee et al., May 6, 2021, “Devices, Systems, and Methods for Treatment of Intracranial Aneurysms”) and 20210153872 (Nguyen et al., May 27, 2021, “Devices, Systems, and Methods for Treatment of Intracranial Aneurysms”) disclose delivering an occlusive member to an aneurysm cavity via an elongated shaft and transforming a shape of the occlusive member within the cavity and introducing an embolic element to a space between the occlusive member and an inner surface of the aneurysm wall.

U.S. patent application 20180303486 (Rosenbluth et al., Oct. 25, 2018, “Embolic Occlusion Device and Method”) discloses an occlusion device including a tubular braided member with a repeating pattern of larger diameter portions and smaller diameter portions along a longitudinal axis. U.S. patent application 20160022445 (Ruvalcaba et al., Jan. 28, 2016, “Occlusive Device”) and 20190343664 (Ruvalcaba et al., Nov. 14, 2019, “Occlusive Device”) disclose an aneurysm embolization device can with a body having a single, continuous piece of material that is shape set into a plurality of distinct structural components and an atraumatic tip portion,

U.S. Pat. No. 8,597,320 (Sepetka et al., Dec. 3, 2013, “Devices and Methods for Treating Vascular Malformations”) discloses a device with a closed mesh structure with a proximal collar and a distal collar, with flexible filaments extending therebetween. U.S. patent application 20190274691 (Sepetka et al., Sep. 12, 2019, “Occlusive Device”) and U.S. Pat. No. 11,045,203 (Sepetka et al., Jun. 29, 2021, “Occlusive Device”) disclose multiple sequentially deployed occlusive devices that are connected together to create an extended length. U.S. Pat. No. 10,729,447 (Shimizu et al., Aug. 4, 2020, “Devices for Vascular Occlusion”) discloses a wide variety of occlusive devices, delivery systems, and manufacturing methods for such devices. U.S. patent applications 20200375607 (Soto Del Valle et al., Dec. 3, 2020, “Aneurysm Device and Delivery System”) and 20200397447 (Lorenzo et al., Dec. 24, 2020, “Aneurysm Device and Delivery System”) disclose a mesh ball in a mesh bowl.

U.S. patent application 20200187952 (Walsh et al., Jun. 18, 2020, “Intrasaccular Flow Diverter for Treating Cerebral Aneurysms”) discloses implants with a stabilizing frame for anchoring and an occluding element for diverting blood flow from an aneurysm neck. U.S. patent application 20200405347 (Walzman, Dec. 31, 2020, “Mesh Cap for Ameliorating Outpouchings”) discloses a self-expandable occluding device can both cover the neck of an outpouching and serve as a permanent embolic plug thereby immediately stabilizing the outpouching.

U.S. Pat. No. 10,398,441 (Warner et al., Sep. 3, 2019, “Vascular Occlusion”) discloses a vascular disorder treatment system comprising a delivery tube, a containment device, a pusher distally movable through a lumen, and a stopper ring. U.S. patent application 20210045750 (Wolf et al., Feb. 18, 2021, “Systems and Methods for Treating Aneurysms”) and U.S. Pat. No. 10,856,880 (Badruddin et al., Dec. 8, 2020, “Systems and Methods for Treating Aneurysms”) disclose an implantable vaso-occlusive device with a proximal end configured to seat against the aneurysm adjacent the neck of the aneurysm and a distal end configured to extend in the sac and away from the neck of the aneurysm.

### SUMMARY OF THE INVENTION

Disclosed herein is an intrasacular aneurysm occlusion device comprising a flexible net or mesh with a proximal portion and a distal portion which is inserted into and expanded within an aneurysm sac. The proximal portion is stiffer and/or less flexible than the distal portion. There are several reasons why it can be advantageous for the flexible net or mesh to have a relatively-stiff proximal portion which covers the aneurysm neck and a relatively-flexible distal portion which fills the aneurysm dome. For example, a relatively-stiff proximal portion is less likely to prolapse into the parent vessel. Also, a relatively-flexible distal portion is more likely to conform to the walls of even an irregularly-shaped aneurysm sac. Further, a relatively-flexible distal portion enables the same size device to fill aneurysms with a wider range of sizes and shapes, thereby reducing inventory costs for multiple size devices. The device also includes an opening through the proximal portion of the net or mesh through which embolic members and/or embolic material is inserted into the flexible net or mesh. Insertion of embolic members and/or material into the flexible net or mesh causes the flexible net or mesh to expand and conform to the walls of even an irregularly-shaped aneurysm sac. The device also includes a closure mechanism which closes the opening after embolic members and/or material has been inserted into the flexible net or mesh.

### BRIEF INTRODUCTION TO THE FIGURES

FIGS. 1 through 4 show views at four different times of an intrasacular aneurysm occlusion device comprising a net or mesh with a relatively-stiff proximal portion which covers an aneurysm neck and relatively-flexible distal portion which fills the aneurysm dome.

FIG. 1 shows this device as the net or mesh is exiting the catheter and being inserted into an aneurysm.

FIG. 2 shows this device after the net or mesh has exited the catheter and been radially expanded to a first extent within the aneurysm.

FIG. 3 shows this device as embolic members and/or embolic material is being delivered into the net or mesh.

FIG. 4 shows this device after the net or mesh has been further expanded by being filled with embolic members and/or embolic material, wherein the further-expanded net or mesh conforms to the contours of the irregularly-shaped aneurysm sac.

### DETAILED DESCRIPTION OF THE FIGURES

In an example, an intrasacular aneurysm occlusion device can comprise: a flexible net or mesh which is inserted into and expanded within an aneurysm sac; wherein the flexible net or mesh further comprises a proximal portion whose centroid is a first distance from the aneurysm neck after expansion within the aneurysm sac; and wherein the proximal portion has a first average level of flexibility (and/or elasticity, stretchability, conformability, pliability, or softness) and a first average level of stiffness (and/or Young's-modulus, resiliency, strength, or durometer); wherein the flexible net or mesh further comprises a distal portion whose centroid is a second distance from the aneurysm neck after expansion within the aneurysm sac; wherein the distal portion has a second average level of flexibility (and/or elasticity, stretchability, conformability, pliability, or softness) and a second average level of stiffness (and/or Young's-modulus, resiliency, strength, or durometer); wherein the first distance is less than the second distance, and wherein (a) the first average level of flexibility (and/or elasticity, stretchability, conformability, pliability, or softness) is less than the second average level of flexibility (and/or elasticity, stretchability, conformability, pliability, or softness) and/or (b) the first average level of stiffness (and/or Young's-modulus, resiliency, strength, or durometer) is greater than the second average level of stiffness (and/or Young's-modulus, resiliency, strength, or durometer); an opening through the proximal portion of the flexible net or mesh; embolic members and/or embolic material which is inserted through the opening into the flexible net or mesh, wherein insertion of the embolic members and/or material into the flexible net or mesh causes the flexible net or mesh to expand and conform to the walls of even an irregularly-shaped aneurysm sac; and a closure mechanism which closes the opening after embolic members and/or material has been inserted through the opening into the flexible net or mesh.

The “longitudinal axis” of a flexible net or mesh can be defined as the longest axis of the net or mesh as it is delivered through a catheter to an aneurysm sac. This axis can still be referred to as the “longitudinal axis” even if the net or mesh is later longitudinally-contracted and radially-expanded (e.g. into a generally spherical or ellipsoidal shape) after insertion into the aneurysm sac. A “lateral plane” can be defined as a virtual plane which is orthogonal to the longitudinal axis. The “mid-section lateral plane” can be defined as the “lateral plane” which bisects the “longitudinal axis.” The “proximal third lateral plane” and the “distal third lateral plane” can be defined as the lateral planes which trisect the longitudinal axis, wherein the proximal plane is closer to the aneurysm neck and the distal plan is farther from the aneurysm neck after the net or mesh has been expanded within the aneurysm sac.

For this disclosure, the default definition of the “proximal portion” of a flexible net or mesh is the portion of the net or mesh which is closer to the aneurysm neck than the “mid-section lateral plane” and the default definition of the “distal portion” of the net or mesh is the portion of the net or mesh which is farther from the aneurysm neck than the “mid-section lateral plane” after the net or mesh has been expanded in an aneurysm sac. Alternatively, a “proximal portion” of a flexible net or mesh may be defined as the portion of the net or mesh which is closer to the aneurysm neck than the “proximal third lateral plane” and a “distal portion” of the net or mesh can be defined as the portion of the net or mesh which is farther from the aneurysm neck than the “distal third lateral plane” after the net or mesh has been expanded in an aneurysm sac. Examples with the alternative definitions of proximal and distal may be understood as variations on examples disclosed herein.

Also with respect definitions and semantics, there are words or phrases which are related to the terms “flexible” and “flexibility” which are either synonyms or which measure physical attributes which are highly-correlated to flexibility. Some of these related words or phrases are: low-Young's-modulus, elastic, stretchable, conformable, compliant, pliable, soft, and low-durometer. It is to be understood that these related words can be substituted for the words “flexible” and “flexibility” in the examples of devices which are disclosed herein, especially as they relate to a proximal portion of a device. Also, there are words or phrases which are related to the terms “stiff,” “stiffer,” and “stiffness” which are either synonyms or which measure physical attributes which are highly-correlated with stiffness. Some of these related words or phrases are: high-Young's-modulus, resilient, strong, and high-durometer. It is to be understood that these related words or phrases can be substituted for the words “stiff,” “stiffer,” and “stiffness” in the examples of nets or meshes which are disclosed herein, especially as they relate to a distal portion of a net or mesh.

There are several reasons why it can be advantageous for a flexible net or mesh which is used for aneurysm occlusion to have a relatively-stiff proximal portion which covers the aneurysm neck and a relatively-flexible distal portion which fills the aneurysm dome. For example, a relatively-stiff proximal portion is less likely to prolapse into the parent vessel. Also, a relatively-flexible distal portion is more likely to conform to the walls of even an irregularly-shaped aneurysm sac. Further, a relatively-flexible distal portion enables the same size device to fill aneurysms with a wider range of sizes and shapes, thereby reducing inventory costs for multiple size devices.

In an example, a flexible, expandable, and liquid-permeable net or mesh can be inserted into and to fit within an aneurysm. In an example, a flexible net or mesh enclosure can receive and retain a plurality of fill members. In an example, a resulting accumulation of the plurality of fill members within the flexible net or mesh can cause the flexible net or mesh to expand and to come into contact with and generally conform to an interior wall of an aneurysm sac. This can substantially occlude the aneurysm and retain the net or mesh within the aneurysm. In an example, a flexible net or mesh can have non-uniform tensile strength, flexibility, plasticity, or elasticity. In an example, a flexible net or mesh can be stronger near one location and less strong but more flexible near another location.

A flexible net or mesh need not be of uniform tensile strength, flexibility, plasticity, or elasticity. It can be more flexible at one or more locations. In an example, a flexible net or mesh can comprise a high-flexibility distal portion and a low-flexibility proximal portion. In an example, a flexible net or mesh can have a distal portion with a first level of flexibility and a proximal portion with a second level of flexibility, wherein the second level is less than the first level. In an example, a flexible net or mesh can have a distal portion with a first level of elasticity and a proximal portion with a second level of elasticity, wherein the second level is less than the first level. In an example, the high-flexibility distal portion of a flexible net or mesh can have an irregular expanded arcuate three-dimensional shape which conforms to the walls of an irregularly-shaped aneurysm sac, while the low-flexibility proximal portion prevents the expandable member from protruding out of the aneurysm sac.

In an example, a flexible net or mesh can comprise a resilient compression-resistant proximal portion. In an example, a flexible net or mesh can be selected from the group consisting of: net; mesh; lattice; balloon; bag; and liner. In an example, a flexible net or mesh can have a shape selected from the group consisting of: apple shape; bowl shape; compress-sphere shape; cylinder; disk; doughnut shape; egg shape; ellipsoid; Frisbee™ shape; frustum; hourglass shape; oval; peanut shape; pear shape; pumpkin shape; ring shape; Saturn shape; sphere; tire shape; and torus. In an example, a flexible net or mesh can have an irregular expanded arcuate three-dimensional shape which conforms to the walls of an irregularly-shaped aneurysm sac.

In an example, a flexible net or mesh can have a low-flexibility proximal portion and a high-flexibility distal portion. In an example, a resilient compression-resistant proximal portion of a flexible net or mesh can further comprise a mesh, network, lattice, or radial array of wires or other stiff fibers. In an example, a resilient compression-resistant proximal portion of a flexible net or mesh can be reinforced with wires or other stiff fibers in order to prevent the expandable member from lapsing out of the aneurysm sac.

In an example, a proximal portion of a flexible net or mesh can comprise a resilient wider-than-neck portion with a first density level and a distal portion of this flexible net or mesh can comprise a flexible sac-filling portion with a second density level, wherein the second level is less than the first level. In an example, the proximal portion of a flexible net or mesh can comprise a resilient wider-than-neck portion with a first elasticity level and a distal portion of this flexible net or mesh can comprise a flexible sac-filling portion with a second elasticity level, wherein the second level is greater than the first level. In an example, the proximal portion of a flexible net or mesh can comprise a resilient wider-than-neck portion with a first flexibility level and a distal portion of this flexible net or mesh can comprise a flexible sac-filling portion with a second flexibility level, wherein the second level is greater than the first level.

In an example, a flexible net or mesh can be braided. In an example, different portions, segments, bulges, or undulations of a flexible net or mesh can have different braid patterns. In an example, a proximal portion, segment, or undulation of a flexible net or mesh can have a first braid pattern and a distal portion, segment, or undulation of this device can have a second braid pattern. In an example, different portions, segments, bulges, or undulations of a flexible net or mesh can have different braid densities. In an example, a proximal portion, segment, or undulation of a flexible net or mesh can have a higher braid density than a distal portion, segment, or undulation of this device. In an example, different portions, segments, bulges, or undulations of a flexible net or mesh can have different braid angles. In an example, a proximal portion, segment, or undulation of a flexible net or mesh can have a greater braid angle than a distal portion, segment, or undulation of this device.

In an example, different portions, segments, bulges, or undulations of a flexible net or mesh can have different braid pitches. In an example, a proximal portion, segment, or undulation of a flexible net or mesh can have a first braid pitch and a distal portion, segment, or undulation of this device can have a second braid pitch. In an example, different portions, segments, bulges, or undulations of a flexible net or mesh can have different braid filament sizes. In an example, a proximal portion, segment, or undulation of a flexible net or mesh can have a first braid filament size and a distal portion, segment, or undulation of this device can have a second braid filament size.

In an example, an intrasacular aneurysm occlusion device can comprise: a flexible net or mesh which is inserted into and expanded within an aneurysm sac; wherein the flexible net or mesh further can comprise a proximal portion whose centroid is a first distance from the aneurysm neck after expansion within the aneurysm sac; and wherein the proximal portion has a first average level of flexibility and a first average level of stiffness; wherein the flexible net or mesh further can comprise a distal portion whose centroid is a second distance from the aneurysm neck after expansion within the aneurysm sac; wherein the distal portion has a second average level of flexibility and a second average level of stiffness; wherein the first distance is less than the second distance; wherein the first average level of flexibility is less than the second average level of flexibility and/or the first average level of stiffness is greater than the second average level of stiffness; an opening through the proximal portion of the flexible net or mesh; embolic members and/or embolic material which is inserted through the opening into the flexible net or mesh, wherein insertion of the embolic members and/or material into the flexible net or mesh causes the flexible net or mesh to expand and conform to the walls of even an irregularly-shaped aneurysm sac; and a closure mechanism which closes the opening after embolic members and/or material has been inserted through the opening into the flexible net or mesh.

In an example, elasticity, stretchability, conformability, pliability, or softness can be substituted for flexibility as a measured characteristic of the proximal and distal portions of the net or mesh. In an example, Young's Modulus, resiliency, strength, or durometer can be substituted for stiffness as a measured characteristic of the proximal and distal portions of the net or mesh.

In an example, the stiffness of the proximal portion of the net or mesh relative to that of the distal portion of the net or mesh can be increased by using thicker wires, tubes, filaments, and/or strands for the proximal portion than those used for the distal portion. In an example, the stiffness of the proximal portion of the net or mesh relative to that of the distal portion of the net or mesh can be increased by using stiffer wires, tubes, filaments, and/or strands for the proximal portion than those used for the distal portion. In an example, the stiffness of the proximal portion of the net or mesh relative to that of the distal portion of the net or mesh can be increased by creating a greater density of wires, tubes, filaments, and/or strands in the proximal portion than in the distal portion.

In an example, the stiffness of the proximal portion of the net or mesh relative to that of the distal portion of the net or mesh can be increased by using a first proportion of metal relative to polymer to create the proximal portion and using a second proportion of metal relative to polymer to create the distal portion, wherein the second proportion is less than the first proportion. In an example, the stiffness of the proximal portion of the net or mesh relative to that of the distal portion of the net or mesh can be increased by adding radial spokes or struts to the proximal portion, wherein a radial array of wires, tubes, or struts extend radially-outward from a central area of the proximal portion of the net or mesh.

In an example, the stiffness of the proximal portion of the net or mesh relative to that of the distal portion can be increased by integrating an array of nested wire rings into the proximal portion. In an example, the stiffness of the proximal portion of the net or mesh relative to that of the distal portion can be increased by integrating an undulating ring of wire into the proximal portion. In an example, the stiffness of the proximal portion of the net or mesh relative to that of the distal portion can be increased by integrating a helical wire into the proximal portion of the net or mesh.

In an example, the stiffness of the proximal portion of the net or mesh relative to that of the distal portion can be increased by integrating one or more coils into the proximal portion of the net or mesh. In an example, the stiffness of the proximal portion of the net or mesh relative to that of the distal portion can be increased by coating wires, tubes, filaments, and/or strands in the proximal portion of the net or mesh with a stiffening material. In an example, the stiffness of the proximal portion of the net or mesh relative to that of the distal portion can be increased by using stiffer material, such as material with a higher Young's Modulus and/or durometer, to create the proximal portion than to create the distal portion.

In an example, the stiffness of the proximal portion of the net or mesh relative to that of the distal portion of the net or mesh can be increased by having a greater number of layers in the proximal portion than in the distal portion. In an example, the flexible net or mesh can comprise a convex spherical, ellipsoidal, and/or generally-globular mesh at least partially within the concavity of a proximal concave mesh with a distal-facing concavity.

In an example, the flexible net or mesh can comprise a convex spherical, ellipsoidal, and/or generally-globular mesh made primarily or entirely from a polymer and at least partially within the concavity of a proximal concave mesh with a distal-facing concavity made primarily or entirely from metal.

In an example, the flexible net or mesh can be made by 3D printing. In an example, the flexible net or mesh can be made by 3D printing with a flexible polymer, wherein the proximal portion of the net or mesh is thicker than the distal portion of the net or mesh. In an example, the flexible net or mesh can be made by 3D printing with a flexible polymer, wherein the proximal portion of the net or mesh is printed with a stiffer polymer than the distal portion of the net or mesh.

FIGS. 1 through 4 show views at four different times of an intrasacular aneurysm occlusion device comprising: a flexible net or mesh which is inserted into and expanded within an aneurysm sac 101; wherein the flexible net or mesh further comprises a proximal portion 102 whose centroid is a first distance from the aneurysm neck after expansion within the aneurysm sac; and wherein the proximal portion has a first average level of flexibility (and/or elasticity, stretchability, conformability, pliability, or softness) and a first average level of stiffness (and/or Young's-modulus, resiliency, strength, or durometer); wherein the flexible net or mesh further comprises a distal portion 103 whose centroid is a second distance from the aneurysm neck after expansion within the aneurysm sac; wherein the distal portion has a second average level of flexibility (and/or elasticity, stretchability, conformability, pliability, or softness) and a second average level of stiffness (and/or Young's-modulus, resiliency, strength, or durometer); wherein the first distance is less than the second distance, and wherein (a) the first average level of flexibility (and/or elasticity, stretchability, conformability, pliability, or softness) is less than the second average level of flexibility (and/or elasticity, stretchability, conformability, pliability, or softness) and/or (b) the first average level of stiffness (and/or Young's-modulus, resiliency, strength, or durometer) is greater than the second average level of stiffness (and/or Young's-modulus, resiliency, strength, or durometer); an opening 104 through the proximal portion of the flexible net or mesh; embolic members and/or embolic material 107 which is inserted through the opening into the flexible net or mesh, wherein insertion of the embolic members and/or material into the flexible net or mesh causes the flexible net or mesh to expand and conform to the walls of even an irregularly-shaped aneurysm sac; and a closure mechanism 105 which closes the opening after embolic members and/or material has been inserted through the opening into the flexible net or mesh. FIGS. 1 through 4 also show a catheter 106 through which the flexible net or mesh is delivered to the aneurysm sac.

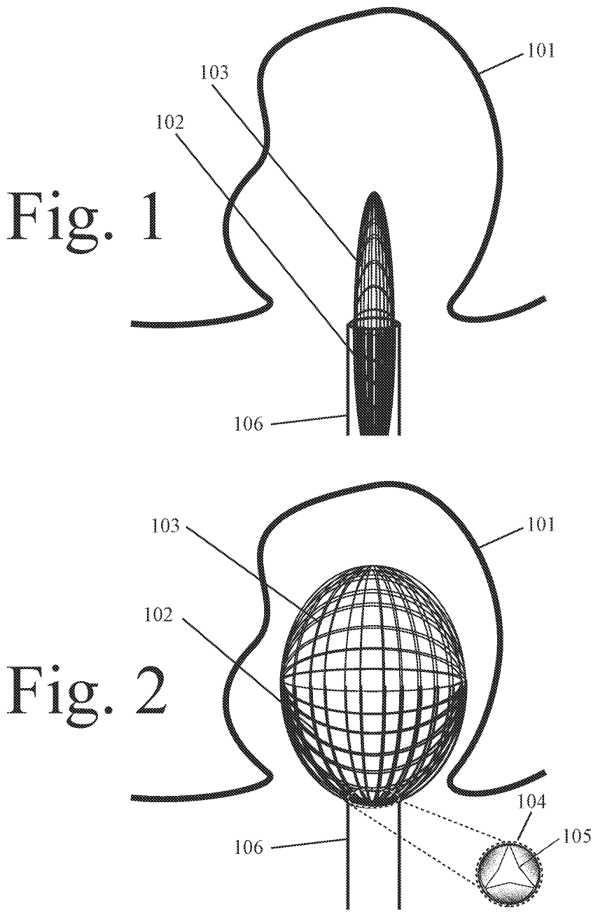

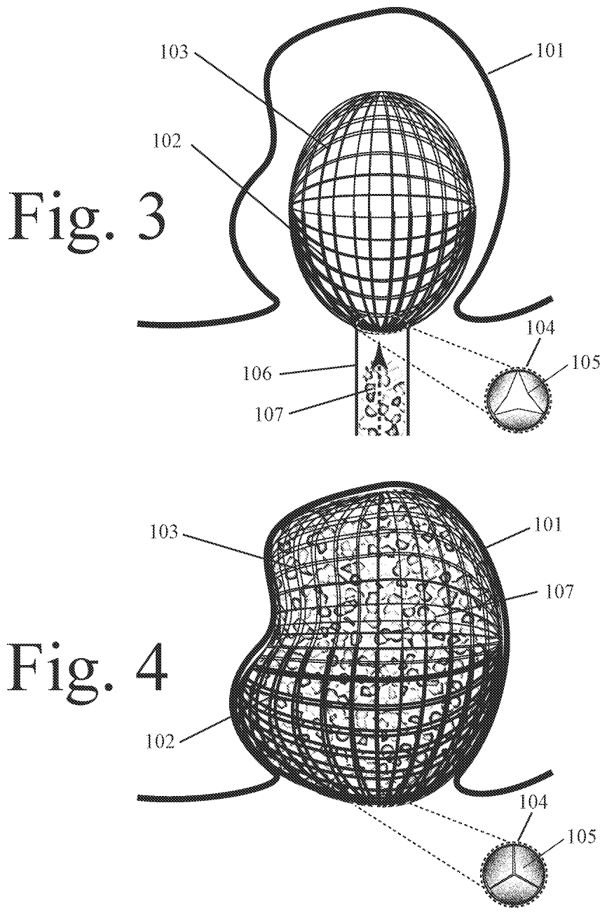

FIG. 1 shows this device at a first point in time, wherein the flexible net or mesh is exiting the catheter and being inserted into an irregularly-shaped aneurysm sac. FIG. 2 shows this device at a second point in time, wherein the flexible net or mesh has exited the catheter and has radially expanded to a first extent within the aneurysm sac. FIG. 3 shows this device at a third point in time, wherein embolic members and/or embolic material is being delivered through the catheter for insertion through the opening into the flexible net or mesh. FIG. 4 shows this device at a fourth point in time, wherein the flexible net or mesh has been further expanded to a second extent by being filled with embolic members and/or embolic material, wherein the further-expanded flexible net or mesh conforms to the contours of the irregularly-shaped aneurysm sac, wherein the closure mechanism has closed the opening through the flexible net or mesh so that the embolic members and/or embolic material does not escape the flexible net or mesh, and wherein the catheter has been detached from the flexible net or mesh and withdrawn from the person's body. In an example, a flexible net or mesh can have: a spherical, ellipsoidal, generally-globular, hemispherical, bowl-shaped, “ball in a bowl,” or hourglass-shaped first configuration when it is first formed; a radially-constrained second configuration as it is delivered through a catheter into an aneurysm sac; and an expanded third configuration after it has been expanded by insertion of embolic members and/or embolic material in an aneurysm sac. In an example, the expanded third configuration can be an irregular shape which conforms to the walls of even an irregularly-shaped aneurysm sac.

In an example, a flexible net or mesh can have a single-layer hemispherical and/or bowl-shaped first configuration which is formed by radially-constraining the proximal end of a tubular mesh with a ring, band, and/or cylinder. In an example, a flexible net or mesh can have a double-layer hemispherical and/or bowl-shaped first configuration which formed by radially-constraining a mid-section of a tubular mesh and then everting a proximal portion of the tubular mesh over a distal portion of the tubular mesh. In an example, a flexible net or mesh can have a double-layer hemispherical and/or bowl-shaped first configuration which is formed by radially-constraining the proximal end of a tubular mesh by a proximal annular member, radially-constraining the distal end of the tubular mesh by a distal annual member, and then inverting the distal portion of the tubular mesh into the concavity of the proximal portion of the tubular mesh. In an example, a flexible net or mesh can have a double-layer hemispherical and/or bowl-shaped first configuration which is formed by radially-constraining both the proximal end and distal ends of a tubular mesh by a proximal member, thereby inverting the distal portion of the tubular mesh into the concavity of the proximal portion of the tubular mesh.

In an example, a proximal portion of a flexible net or mesh can be made from one or more metals and a distal portion of a flexible net or mesh can be made from one or more polymers. In an example, a proximal portion of a flexible net or mesh can be made from Nitinol. In an example, a proximal portion of a net or mesh can be a flexible metal mesh. In an example, a proximal portion of a flexible net or mesh can be a braided metal mesh. In an example, a proximal portion of a flexible net or mesh can be woven or braided from metal filaments, wires, or tubes. In an example, a proximal portion of a flexible net or mesh can be made from shape-memory material.

In an example, a distal portion of a flexible net or mesh can be made from a polymer. In an example, a distal portion of a flexible net or mesh can be woven or braided from polymer threads, filaments, yarns, or strips. In an example, a distal portion of a flexible net or mesh can be 3D printed. In an example, a distal portion of a flexible net or mesh can be made from an elastic and/or stretchable polymer. In an example, a distal portion of a flexible net or mesh can be elastic and/or stretchable and can expand as it is filled with embolic members and/or material. In an example, a distal portion of a flexible net or mesh can be sufficiently flexible to conform to the shape of even an irregularly-shaped aneurysm sac as the net or mesh is filled with embolic members and/or material. In an example, a distal portion of a flexible net or mesh can be sufficiently flexible to conform to the shape of even an irregularly-shaped (e.g. non-spherical) aneurysm sac as the net or mesh is filled with embolic members and/or material. In an example, a distal portion of a flexible net or mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

Alternatively, both the proximal and distal portions of a flexible net or mesh can be made from one or more metals. In an example, the proximal and distal portions of a flexible net or mesh can be made from Nitinol. In an example, the proximal and distal portions can be flexible metal mesh. In an example, the proximal and distal portions of a flexible net or mesh can be a braided metal mesh. In an example, the proximal and distal portions of a flexible net or mesh can be woven or braided from metal filaments, wires, or tubes. In an example, the proximal and distal portions of a flexible net or mesh can be made from shape-memory material.

Alternatively, both the proximal and distal portions of a flexible net or mesh can be made from one or more polymers. In an example, the proximal and distal portions of a flexible net or mesh can be woven or braided from polymer threads, filaments, yarns, or strips. In an example, the proximal and distal portions of a flexible net or mesh can be 3D printed. In an example, the proximal and distal portions of a flexible net or mesh can be made from an elastic and/or stretchable polymer. In an example, the proximal and distal portions of a flexible net or mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, the proximal and/or distal portions of a net or mesh can be made from polycarbonate urethane (PCU). In an example, the proximal and/or distal portions of a net or mesh can be made from polydimethylsiloxane (PDMS). In an example, the proximal and/or distal portions of a net or mesh can be made from polyesters. In an example, the proximal and/or distal portions of a net or mesh can be made from polyether block amide (PEBA). In an example, the proximal and/or distal portions of a net or mesh can be made from polyetherether ketone (PEEK). In an example, the proximal and/or distal portions of a net or mesh can be made from polyethylene. In an example, the proximal and/or distal portions of a net or mesh can be made from polyethylene glycol (PEG). In an example, the proximal and/or distal portions of a net or mesh can be made from polyethylene terephthalate (PET).

In an example, the proximal and/or distal portions of a net or mesh can be made from polyglycolic acid (PGA). In an example, the proximal and/or distal portions of a net or mesh can be made from polylactic acid (PLA). In an example, the proximal and/or distal portions of a net or mesh can be made from poly-N-acetylglucosamine (PNAG). In an example, the proximal and/or distal portions of a net or mesh can be made from polyolefin. In an example, the proximal and/or distal portions of a net or mesh can be made from polypropylene. In an example, the proximal and/or distal portions of a net or mesh can be made from polytetrafluoroethylene (PTFE). In an example, the proximal and/or distal portions of a net or mesh can be made from polyurethane (PU). In an example, the proximal and/or distal portions of a net or mesh can be made from polyvinyl alcohol (PVA). In an example, the proximal and/or distal portions of a net or mesh can be made from polyvinyl pyrrolidone (PVP).

In an example, pores or holes in a flexible net or mesh can be smaller than the size (e.g. diameter, width, and/or length) of embolic members and/or material which is inserted into the net or mesh so that the embolic members and/or material do not escape out of the net or mesh. In an example, pores or holes in a flexible net or mesh can less than half of the size (e.g. diameter, width, and/or length) of embolic members and/or material which is inserted into the net or mesh so that the embolic members and/or material do not escape out of the net or mesh. In an example, pores or holes in a flexible net or mesh can have a size which is less than half of the smallest diameter and/or width of embolic members and/or material which is inserted into the net or mesh so that the embolic members and/or material do not escape out of the net or mesh. In an example, pores or holes in a flexible net or mesh can have a size which less than half of the smallest length of embolic members and/or material which is inserted into the net or mesh so that the embolic members and/or material do not escape out of the net or mesh.

In an example, a net or mesh can have hexagonal pores. In an example, a net or mesh with hexagonal pores can be made using 3D printing. In an example, a flexible metal net or mesh with hexagonal pores can be made by 3D printing with liquid metal. In an example, a net or mesh with hexagonal pores can be made by 3D printing with a polymer. In an example, a net or mesh with hexagonal pores can be made by 3D printing with an elastomeric polymer. In an example, a net or mesh with hexagonal pores can be made by 3D printing with a silicone-based polymer. In an example, a net or mesh with hexagonal pores can be made by 3D printing with polydimethylsiloxane (PDMS).

In an example, a net or mesh can have quadrilateral pores. In an example, a net or mesh with quadrilateral pores can be made using 3D printing. In an example, a flexible metal net or mesh with quadrilateral pores can be made by 3D printing with liquid metal. In an example, a net or mesh with quadrilateral pores can be made by 3D printing with a polymer. In an example, a net or mesh with quadrilateral pores can be made by 3D printing with an elastomeric polymer. In an example, a net or mesh with quadrilateral pores can be made by 3D printing with a silicone-based polymer. In an example, a net or mesh with quadrilateral pores can be made by 3D printing with polydimethylsiloxane (PDMS).

In an example, a net or mesh can have circular pores. In an example, a net or mesh with circular pores can be made using 3D printing. In an example, a flexible metal net or mesh with circular pores can be made by 3D printing with liquid metal. In an example, a net or mesh with circular pores can be made by 3D printing with a polymer. In an example, a net or mesh with circular pores can be made by 3D printing with an elastomeric polymer. In an example, a net or mesh with circular pores can be made by 3D printing with a silicone-based polymer. In an example, a net or mesh with circular pores can be made by 3D printing with polydimethylsiloxane (PDMS).

In an example, a net or mesh can be made with a cobalt chromium alloy. In an example, a net or mesh can be made with a nickel-titanium alloy. In an example, a net or mesh can comprise cobalt chromium alloy wires, filaments, or tubes. In an example, a net or mesh can comprise nickel-titanium alloy wires, filaments, or tubes. In an example, a net or mesh can comprise nitinol wires, filaments, or tubes. In an example, a net or mesh can be made with nitinol. In an example, a net or mesh can comprise platinum wires, filaments, or tubes. In an example, a net or mesh can be made with platinum. In an example, a net or mesh can comprise stainless steel wires, filaments, or tubes. In an example, a net or mesh can be made with stainless steel. In an example, a net or mesh can comprise tantalum wires, filaments, or tubes. In an example, a net or mesh can be made with tantalum.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a net or mesh can be transformed into a single-layer ellipsoidal and/or generally globular flexible net or mesh by two annular members which radially-constrain the proximal and distal ends of a net or mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a net or mesh is transformed into single-layer spherical flexible net or mesh by two annular members which radially-constrain the proximal and distal ends of a net or mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be transformed into a single-layer, distally-concave, bowl-shaped flexible net or mesh by a single annular member which radially-constrains the proximal end of a tubular mesh. In an example, a tubular mesh can be transformed into single-layer, proximally-concave, bowl-shaped flexible net or mesh by a single annular member which radially-constrains the distal end of a tubular mesh.

In an example, a tubular mesh can be transformed into a double-layer, distally-concave, bowl-shaped flexible net or mesh by two annular members which radially-constrain the proximal and distal ends of a tubular mesh, wherein the distal portion of a tubular mesh is inverted proximally (e.g. folded proximally) until it has a distally-concave shape. In an example, the distal circumference of the flexible net or mesh is a fold in the net or mesh. In an example, both of the radially-constrained ends can project into the interior of flexible net or mesh. In an example, the proximal end can be inverted to project into the interior of bowl-shaped flexible net or mesh and the distal end is not. Alternatively, a tubular mesh can be transformed into double-layer, distally-concave, bowl-shaped flexible net or mesh by a single annular member in a middle section (between the ends) of a tubular mesh which radially-constrains the middle of a tubular mesh, wherein the proximal portion of a tubular mesh is everted distally until it has a distally-concave shape. In an example, the distal circumference of a flexible net or mesh can comprise two nested tubular openings.

In an example, a tubular mesh from which a flexible net or mesh is formed can be tapered. In an example, the distal end of a tubular mesh can have a smaller diameter than the proximal end of a tubular mesh. In an example, the distal end of a tubular mesh can have a larger diameter than the proximal end of a tubular mesh. In an example, a tubular mesh from which a flexible net or mesh is formed can have differential flexibility. In an example the distal portion of a tubular mesh can have a first level of flexibility and the proximal portion of a tubular mesh can have a second level of flexibility, wherein the first level is less than the second level. In an example the distal portion of a tubular mesh can have a first level of flexibility and the proximal portion of a tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of a tubular mesh can have a second porosity level, wherein the first level is less than the second level. In an example the distal portion of a tubular mesh can have a first porosity level and the proximal portion of a tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of a tubular mesh can have a second durometer level, wherein the first level is less than the second level. In an example the distal portion of a tubular mesh can have a first durometer level and the proximal portion of a tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members. In various examples, a flexible net or mesh can have a post-expansion shape that is selected from the group consisting of spherical, ellipsoidal, toroidal, compressed-sphere shaped, egg shaped, Saturn shaped, hour-glass shaped, peanut shaped, beehive shaped and geodesic.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

There are several material and structural factors which can affect the relative stiffness and flexibility of the proximal and distal portions, respectively, of a flexible net or mesh. These factors can be selected, adjusted, and/or combined during the design and creation of a flexible net or mesh in order to create the desired stiffness (or flexibility) of the proximal portion of the flexible net or mesh relative to the flexibility (or stiffness) of the distal of the net or mesh.

In an example, the stiffness of a proximal portion of the net or mesh relative to that of a distal portion of the net or mesh can be increased by using thicker wires, tubes, filaments, and/or strands for a proximal portion than those used for a distal portion. In an example, the stiffness of a proximal portion of the net or mesh relative to that of a distal portion of the net or mesh can be increased by using stiffer wires, tubes, filaments, and/or strands for a proximal portion than those used for a distal portion. In an example, the stiffness of a proximal portion of the net or mesh relative to that of a distal portion of the net or mesh can be increased by creating a greater density of wires, tubes, filaments, and/or strands in a proximal portion than in a distal portion. In an example, the stiffness of a proximal portion of the net or mesh relative to that of a distal portion of the net or mesh can be increased by using a first proportion of metal relative to polymer to create a proximal portion and using a second proportion of metal relative to polymer to create a distal portion, wherein the second proportion is less than the first proportion.

In an example, the stiffness of a proximal portion of the net or mesh relative to that of a distal portion of the net or mesh can be increased by adding radial spokes or struts to a proximal portion, wherein a radial array of wires, tubes, or struts extend radially-outward from a central area of a proximal portion of the net or mesh. In an example, the stiffness of a proximal portion of the net or mesh relative to that of a distal portion can be increased by integrating an array of nested wire rings into a proximal portion. In an example, the stiffness of a proximal portion of the net or mesh relative to that of a distal portion can be increased by integrating an undulating ring of wire into a proximal portion. In an example, the stiffness of a proximal portion of the net or mesh relative to that of a distal portion can be increased by integrating a helical wire into a proximal portion of the net or mesh.

In an example, the stiffness of a proximal portion of the net or mesh relative to that of a distal portion can be increased by integrating one or more coils into a proximal portion of the net or mesh. In an example, the stiffness of a proximal portion of the net or mesh relative to that of a distal portion can be increased by coating wires, tubes, filaments, and/or strands in a proximal portion of the net or mesh with a stiffening material. In an example, the stiffness of a proximal portion of the net or mesh relative to that of a distal portion can be increased by using stiffer material, such as material with a higher Young's Modulus and/or durometer, to create a proximal portion than to create a distal portion.

In an example, a flexible net or mesh can be made by 3D printing. In an example, a flexible net or mesh can be made by 3D printing with a flexible polymer, wherein a proximal portion of the net or mesh is thicker than a distal portion of the net or mesh. In an example, a flexible net or mesh can be made by 3D printing with a flexible polymer, wherein a proximal portion of the net or mesh is printed with a stiffer polymer than a distal portion of the net or mesh.

In an example, the stiffness of a proximal portion of the net or mesh relative to that of a distal portion of the net or mesh can be increased by having a greater number of layers in a proximal portion than in a distal portion. In an example, a flexible net or mesh can comprise a convex spherical, ellipsoidal, and/or generally-globular mesh at least partially within the concavity of a proximal concave mesh with a distal-facing concavity. In an example, a flexible net or mesh can comprise a convex spherical, ellipsoidal, and/or generally-globular mesh made primarily or entirely from a polymer and at least partially within the concavity of a proximal concave mesh with a distal-facing concavity made primarily or entirely from metal.

In an example, the stiffness of a proximal portion of the net or mesh can be increased by using thick wires, tubes, filaments, and/or strands in the proximal portion. In an example, the stiffness of a proximal portion of the net or mesh can be increased by using stiff wires, tubes, filaments, and/or strands in the proximal portion. In an example, the stiffness of a proximal portion of the net or mesh can be increased by creating a high density of wires, tubes, filaments, and/or strands in the proximal portion. In an example, the stiffness of a proximal portion of the net or mesh can be increased by using a high proportion of metal relative to polymer to create the proximal portion.

In an example, the stiffness of a proximal portion of the net or mesh can be increased by adding radial spokes or struts to a proximal portion of a net or mesh, wherein a radial array of wires, tubes, or struts extend radially-outward from a central area of the proximal portion. In an example, the stiffness of a proximal portion of the net or mesh can be increased by integrating an array of nested wire rings into a proximal portion. In an example, the stiffness of a proximal portion of the net or mesh can be increased by integrating an undulating ring of wire into a proximal portion. In an example, the stiffness of a proximal portion of the net or mesh can be increased by integrating a helical wire into a proximal portion of the net or mesh.

In an example, the stiffness of a proximal portion of the net or mesh can be increased by integrating one or more coils into a proximal portion of the net or mesh. In an example, the stiffness of a proximal portion of the net or mesh can be increased by coating wires, tubes, filaments, and/or strands in the proximal portion with a stiffening material. In an example, the stiffness of a proximal portion of the net or mesh can be increased by using stiff material, such as material with a high Young's Modulus and/or durometer, to create the proximal portion. In an example, the stiffness of a proximal portion of the net or mesh can be increased by having multiple layers (e.g. two or more layers) in the proximal portion.

In an example, the stiffness of a proximal portion of a net or mesh relative to that of a distal portion of the net or mesh can be increased by creating a greater number of layers (e.g. two or more mesh layers instead of one) for the proximal portion than for the distal portion. In an example, the stiffness of a proximal portion of a net or mesh relative to that of the distal portion of the net or mesh can be increased by adding a radial-spoke structure to the proximal portion, wherein a radial array of thicker wires, tubes, or struts extend radially outward from the center of the proximal portion of the net or mesh. In an example, the stiffness of a proximal portion of a net or mesh relative to that of the distal portion of the net or mesh can be increased by integrating a radial-spoke wire structure into the proximal portion. In various examples, this can be done by adhesion, melting, weaving, or braiding. In an example, the stiffness of a proximal portion of a net or mesh relative to that of the distal portion of a net or mesh can be increased by coating wires, tubes, and/or strands in the proximal portion of the net or mesh with a polymer coating.

In an example, the Young's modulus of (the material used to make) the distal portion of a net or mesh can be between 1 and 5. In an example, the stiffness (or resiliency, strength, and/or durometer) of a proximal portion of a net or mesh relative to that of a distal portion of the net or mesh can be expressed as a percentage. In another example, the Young's modulus of (the material used to make) the distal portion of a net or mesh can be between 0.005 and 0.01. In one embodiment, the Young's modulus of (the material used to make) the proximal portion of a net or mesh can be greater than 0.1. In another example, the average flexibility of a proximal portion of a net or mesh can be between 33% and 75% of the average flexibility of a distal portion of the net or mesh. Alternatively, the stiffness of the proximal portion of a net or mesh can be greater than 0.001 N/mm and the stiffness of the distal portion of a net or mesh can be less than 0.001 N/mm. In an example, the Young's modulus of (the material used to make) the proximal portion of a net or mesh can be between 1 and 10.

In another example, the Young's modulus of (the material used to make) the proximal portion of a net or mesh can be between 0.005 and 0.01. In an example, the stiffness (or resiliency, strength, and/or durometer) of a distal portion of a net or mesh relative to that of a proximal portion of the net or mesh can be expressed as a percentage. Alternatively, the Young's modulus of (the material used to make) the proximal portion of a net or mesh can be between 0.01 and 0.05. In an example, the Young's modulus of (the material used to make) the proximal portion of a net or mesh can be greater than 1. Alternatively, the average stiffness of a distal portion of a net or mesh can be between one-third and three-quarters that of a proximal portion of the net or mesh. In an example, the stiffness of the proximal portion of a net or mesh can be greater than 0.01 N/mm and the stiffness of the distal portion of a net or mesh can be less than 0.005 N/mm.

In an example, the Young's modulus of (the material used to make) the proximal portion of a net or mesh can be between 5 and 10. In an example, the average flexibility of a proximal portion of a net or mesh can be between one-third and three-quarters that of a distal portion of the net or mesh. In an example, the stiffness of the proximal portion of a net or mesh can be greater than 0.005 N/mm and the stiffness of the distal portion of a net or mesh can be less than 0.005 N/mm. In an example, the Young's modulus of (the material used to make) the proximal portion of a net or mesh can be between 1 and 5. In one embodiment, the stiffness of the distal portion of a net or mesh can be less than 0.025 N/mm. Alternatively, the Young's modulus of (the material used to make) the distal portion of a net or mesh can be between 0.1 and 0.5.

In an example, the stiffness of a proximal portion of a net or mesh relative to that of the distal portion can be increased by using a higher-density mesh for the proximal portion than for the distal portion. In an example, the stiffness of a proximal portion of a net or mesh relative to that of the distal portion can be increased by adhering and/or melting a helical wire structure onto the proximal portion. In an example, the stiffness of a proximal portion of a net or mesh relative to that of the distal portion can be increased by using material with a higher Young's modulus and/or durometer to create the proximal portion than for the distal portion.

In an example, the stiffness of the proximal portion of a net or mesh can be greater than 0.1 N/mm and the stiffness of the distal portion of a net or mesh can be less than 0.05 N/mm. In an example, the Young's modulus of (the material used to make) the proximal portion of a net or mesh can be between 0.001 and 0.01. In an example, the Young's modulus of (the material used to make) the proximal portion of a net or mesh can be greater than 0.01. In an example, the average flexibility of a distal portion of a net or mesh can be at least 200% of average flexibility of a proximal portion of the net or mesh. Alternatively, the stiffness of the distal portion of a net or mesh can be less than 0.05 N/mm. In one embodiment, the Young's modulus of (the material used to make) the proximal portion of a net or mesh can be between 0.1 and 0.5.

In an example, the average stiffness of a proximal portion of a net or mesh can be at least 200% of the average stiffness of a distal portion of the net or mesh. In one embodiment, the Young's modulus of (the material used to make) the proximal portion of a net or mesh can be between 0.1 and 1. In an example, the Young's modulus of (the material used to make) the distal portion of a net or mesh can be less than 0.1. Alternatively, the average stiffness of a proximal portion of a net or mesh can be between 1.5 and 3 times that of a distal portion of the net or mesh. In an example, the Young's modulus of (the material used to make) the distal portion of a net or mesh can be between 0.1 and 1. In an example, the Young's modulus of (the material used to make) the distal portion of a net or mesh can be less than 1.

In an example, the flexibility (or elasticity, stretchability, pliability, and/or softness) of a distal portion of a net or mesh relative to that of a proximal portion of the net or mesh can be expressed as a proportion, ratio, or fraction. In an example, the Young's modulus of (the material used to make) the distal portion of a net or mesh can be between 0.01 and 0.05. Alternatively, the Young's modulus of (the material used to make) the proximal portion of a net or mesh can be greater than 10. In an example, the average flexibility of a distal portion of a net or mesh can be between 1.5 and 3 times that of a proximal portion of the net or mesh. In one embodiment, the Young's modulus of (the material used to make) the distal portion of a net or mesh can be between 0.5 and 1. In an example, the Young's modulus of (the material used to make) the distal portion of a net or mesh can be less than 10.

In an example, the stiffness of a proximal portion of a net or mesh relative to that of the distal portion can be increased by using a tighter braid or weave of wires, tubes, and/or strands to create the proximal portion than for the distal portion. In an example, the stiffness of a proximal portion of a net or mesh relative to that of the distal portion can be increased by adding a “flower petals” shape configuration of structural elements (e.g. large-diameter wires) to the proximal portion of the net or mesh. In an example, the stiffness of a proximal portion of a net or mesh relative to that of the distal portion can be increased by having two layers in the proximal portion of the net or mesh by radially constraining a middle section of a tubular mesh and inverting (or everting) a proximal portion of the net or mesh to create a “ball in a bowl” configuration. In an example, the stiffness of a proximal portion of a net or mesh relative to that of the distal portion can be increased by adding a hub-and-spoke configuration of radial structural elements (e.g. large wires) to the proximal portion of the net or mesh. In an example, the stiffness of a proximal portion of a net or mesh relative to that of the distal portion can be increased by integrating a hub-and-spoke wire structure with the proximal portion.

In an example, the average flexibility of a distal portion of a net or mesh can be between 150% and 300% of the average flexibility of a proximal portion of the net or mesh. Alternatively, the Young's modulus of (the material used to make) the proximal portion of a net or mesh can be between 0.5 and 1. In an example, the Young's modulus of (the material used to make) the proximal portion of a net or mesh can be greater than 0.5 and the Young's modulus of (the material used to make) the distal portion can be less than 0.5. Alternatively, the average stiffness of a proximal portion of a net or mesh can be between 150% and 300% of the average stiffness of a distal portion of the net or mesh. In an example, the Young's modulus of (the material used to make) the distal portion of a net or mesh can be between 1 and 10. In an example, the Young's modulus of (the material used to make) the proximal portion of a net or mesh can be greater than 1 and the Young's modulus of (the material used to make) the distal portion can be less than 1.

In another example, the average flexibility of a proximal portion of a net or mesh can be less than half that of a distal portion of the net or mesh. In one embodiment, the stiffness of the proximal portion of a net or mesh can be greater than 0.025 N/mm and the stiffness of the distal portion of a net or mesh can be less than 0.025 N/mm. In an example, the Young's modulus of (the material used to make) the proximal portion of a net or mesh can be greater than 0.001. Alternatively, the average stiffness of a distal portion of a net or mesh can be less than 50% of the average stiffness of a proximal portion of the net or mesh. In another example, the stiffness of the proximal portion of a net or mesh can be greater than 0.001 N/mm. In an example, the stiffness of the proximal portion of a net or mesh can be greater than 0.025 N/mm and the stiffness of the distal portion of a net or mesh can be less than 0.01 N/mm.

In an example, the flexibility (or elasticity, stretchability, pliability, and/or softness) of a proximal portion of a net or mesh relative to that of a distal portion of the net or mesh can be expressed as a proportion, ratio, or fraction. Alternatively, the stiffness of the distal portion of a net or mesh can be less than 0.001 N/mm. In another example, the Young's modulus of (the material used to make) the distal portion of a net or mesh can be between 0.01 and 0.1. In an example, the stiffness (or resiliency, strength, and/or durometer) of a proximal portion of a net or mesh relative to that of a distal portion of the net or mesh can be expressed as a proportion, ratio, or fraction. In another example, the stiffness of the distal portion of a net or mesh can be less than 0.005 N/mm. In an example, the Young's modulus of (the material used to make) the proximal portion of a net or mesh can be between 0.01 and 0.1.

In an example, the stiffness of a proximal portion of a net or mesh relative to that of the distal portion can be increased by adhering and/or melting a hub-and-spoke wire structure onto the proximal portion. In an example, the stiffness of a proximal portion of a net or mesh relative to that of the distal portion can be increased by using wider-diameter and/or thicker wires, tubes, and/or strands to create the proximal portion than for the distal portion. In an example, the stiffness of a proximal portion of a net or mesh relative to that of the distal portion can be increased by adding a nested ring configuration (e.g. concentric circles) of radial structural elements (e.g. large wires) to the proximal portion of the net or mesh. In an example, the stiffness of a proximal portion of a net or mesh relative to that of the distal portion can be increased by integrating a nested wire rings (e.g. concentric wire rings) with the proximal portion. In an example, the stiffness of a proximal portion of a net or mesh relative to that of the distal portion can be increased by adding a sinusoidal-circular configuration of structural elements (e.g. large wires) to the proximal portion of the net or mesh.

In an example, the stiffness of a proximal portion of a net or mesh relative to that of the distal portion can be increased by integrating an undulating circle of wire onto the proximal portion. In an example, the stiffness of a proximal portion of a net or mesh relative to that of the distal portion can be increased by having two layers in the proximal portion of the net or mesh and only one layer in the distal portion. In an example, the stiffness of a proximal portion of a net or mesh relative to that of the distal portion can be increased by adding a helical (large-diameter wire) structure to the proximal portion of the net or mesh. In an example, the stiffness of a proximal portion of a net or mesh relative to that of the distal portion can be increased by increasing the wire density in the proximal portion of the net or mesh.

In an example, a proximal portion of a flexible net or mesh can be made by braiding or weaving metal wires, tubes, or filaments and a distal portion of the net or mesh can be made by 3D printing with a flexible, elastic, and/or stretchable polymer. In an example, a flexible net or mesh can be made by 3D printing with a flexible polymer, wherein the proximal portion of the printed 3D mesh is thicker than the distal portion of the printed 3D mesh. In an example, a flexible net or mesh can be made by 3D printing with a flexible polymer, wherein the proximal portion of the printed 3D mesh is denser than the distal portion of the printed 3D mesh. In an example, a flexible net or mesh can be made by 3D printing with a flexible polymer, wherein the proximal portion of the printed mesh is printed with a stiffer polymer than the distal portion of the printed mesh. In an example, a flexible net or mesh can be made by 3D printing with a flexible polymer, wherein the proximal portion of the 3D printed mesh has more layers than the distal portion of the 3D printed mesh.

In an example, the stiffness of a proximal portion of a net or mesh relative to that of the distal portion can be increased by having two layers in the proximal portion of the net or mesh by radially constraining and inverting (or everting) a proximal portion of the net or mesh. In an example, the stiffness of a proximal portion of a net or mesh relative to that of the distal portion can be increased by adhering and/or melting a radial-spoke wire structure onto the proximal portion. In an example, the stiffness of a proximal portion of a net or mesh relative to that of the distal portion can be increased by weaving and/or braiding additional large wires into the proximal portion. In an example, the stiffness of a proximal portion of a net or mesh relative to that of the distal portion can be increased by adding a helical (large-diameter wire) coil to the proximal portion of the net or mesh. In an example, the stiffness of a proximal portion of a net or mesh relative to that of the distal portion can be increased by integrating a helical wire structure with the proximal portion.

In an example, the stiffness (or resiliency, strength, and/or durometer) of a distal portion of a net or mesh relative to that of a proximal portion of the net or mesh can be expressed as a proportion, ratio, or fraction. In an example, the average flexibility of a distal portion of a net or mesh can be at least 2 times that of a proximal portion of the net or mesh. In an example, the stiffness of the distal portion of a net or mesh can be less than 0.01 N/mm. In an example, the Young's modulus of (the material used to make) the proximal portion of a net or mesh can be between 0.05 and 0.1. In one embodiment, the average flexibility of a proximal portion of a net or mesh can be less than 50% of the average flexibility of a distal portion of the net or mesh. Alternatively, the stiffness of the proximal portion of a net or mesh can be greater than 0.01 N/mm and the stiffness of the distal portion of a net or mesh can be less than 0.01 N/mm.

In an example, the stiffness of a proximal portion of a net or mesh relative to that of the distal portion can be increased by weaving and/or braiding radial spokes into the proximal portion. In an example, the stiffness of a proximal portion of a net or mesh relative to that of the distal portion can be increased by adhering and/or melting nested wire rings (e.g. concentric wire rings) onto the proximal portion. In an example, the stiffness of a proximal portion of a net or mesh relative to that of the distal portion can be increased by weaving and/or braiding thicker and/or wider-diameter wires, tubes, and/or strands into the mesh of the proximal portion, relative to that of the distal portion. In an example, the stiffness of a proximal portion of a net or mesh relative to that of the distal portion can be increased by coating wires, tubes, and/or strands in the proximal portion of the net or mesh with a stiffening material. In an example, the stiffness of a proximal portion of a net or mesh relative to that of the distal portion can be increased by weaving and/or braiding wires, tubes, and/or strands with a higher Young's modulus and/or durometer into the mesh of the proximal portion, relative to that of the distal portion.

In an example, the stiffness of a proximal portion of a net or mesh relative to that of the distal portion can be increased by having two layers in the proximal portion of the net or mesh by radially constraining a middle section of a tubular mesh and inverting (or everting) a proximal portion of the net or mesh. In an example, the stiffness of a proximal portion of a net or mesh relative to that of the distal portion can be increased by having two layers in the proximal portion of the net or mesh by radially constraining a middle section of the net or mesh and inverting (or everting) a proximal portion of the net or mesh. In an example, the stiffness of a proximal portion of a net or mesh relative to that of the distal portion can be increased by adding large wires or tubes which radiate out from the proximal center of the net or mesh on the proximal portion. In an example, the stiffness of a proximal portion of a net or mesh relative to that of the distal portion can be increased by using a thicker layer of material to create the proximal portion than for the distal portion. In an example, the stiffness of a proximal portion of a net or mesh relative to that of the distal portion can be increased by adding resilient structural elements (e.g. thick radial wires or tubes) to the proximal portion which are not in the distal portion.

In another example, the Young's modulus of (the material used to make) the distal portion of a net or mesh can be between 5 and 10. In an example, the stiffness of the proximal portion of a net or mesh can be greater than 0.01 N/mm. In another example, the stiffness of the proximal portion of a net or mesh can be greater than 0.05 N/mm and the stiffness of the distal portion of a net or mesh can be less than 0.05 N/mm. In one embodiment, the Young's modulus of (the material used to make) the proximal portion of a net or mesh can be between 0.001 and 0.005. In an example, the average stiffness of a distal portion of a net or mesh can be between 33% and 75% of the average stiffness of a proximal portion of the net or mesh. Alternatively, the stiffness of the proximal portion of a net or mesh can be greater than 0.005 N/mm and the stiffness of the distal portion of a net or mesh can be less than 0.001 N/mm.

In an example, a flexible net or mesh whose proximal portion is stiffer than its distal portion can be made by connecting a globular, elastic polymer mesh with a proximal bowl-shaped metal mesh. In an example, a flexible net or mesh whose proximal portion is stiffer than its distal portion can be made by attaching a generally-globular flexible and elastic polymer mesh to a distal-facing concavity of proximal bowl-shaped metal mesh. In an example, a flexible net or mesh can comprise a convex (e.g. spherical, ellipsoidal, and/or generally-globular) polymer mesh and a proximal concave (e.g. hemispherical and/or bowl-shaped) metal mesh. In an example, a flexible net or mesh can be made by combining a convex (e.g. spherical, ellipsoidal, and/or generally-globular) polymer mesh and a proximal concave (e.g. hemispherical and/or bowl-shaped) metal mesh.

In an example, the Young's modulus of (the material used to make) the distal portion of a net or mesh can be between 0.001 and 0.01. In an example, the average stiffness of a proximal portion of a net or mesh can be at least 2 times that of a distal portion of the net or mesh. Alternatively, the Young's modulus of (the material used to make) the distal portion of a net or mesh can be between 0.05 and 0.1. In an example, the Young's modulus of (the material used to make) the distal portion of a net or mesh can be less than 0.01. In another example, the flexibility (or elasticity, stretchability, pliability, and/or softness) of a proximal portion of a net or mesh relative to that of a distal portion of the net or mesh can be expressed as a percentage. In an example, the stiffness of the proximal portion of a net or mesh can be greater than 0.05 N/mm.

In an example, the stiffness of a proximal portion of a net or mesh relative to that of the distal portion can be increased by adding a star-burst configuration of radial structural elements (e.g. large wires) to the proximal portion of the net or mesh. In an example, the stiffness of a proximal portion of a net or mesh relative to that of the distal portion can be increased by integrating one or more coils into the proximal portion of the net or mesh. In an example, the stiffness of a proximal portion of a net or mesh relative to that of the distal portion can be increased by coating wires, tubes, and/or strands in the proximal portion of the net or mesh with an expanding material. In an example, the stiffness of a proximal portion of a net or mesh relative to that of the distal portion can be increased by coating wires, tubes, and/or strands in the proximal portion of the net or mesh with hydrogel material. In an example, the stiffness of a proximal portion of a net or mesh relative to that of the distal portion can be increased by coating wires, tubes, and/or strands in the proximal portion of the net or mesh with a metal coating. In an example, the stiffness of a proximal portion of a net or mesh relative to that of the distal portion can be increased by adhering and/or melting an undulating circle of wire onto the proximal portion (and it burns, burns, burns, the ring of wire, the ring of wire).

In an example, the average stiffness of a distal portion of a net or mesh can be less than half that of a proximal portion of the net or mesh. In one embodiment, the stiffness of the proximal portion of a net or mesh can be greater than 0.005 N/mm. In an example, the stiffness of the proximal portion of a net or mesh can be greater than 0.05 N/mm and the stiffness of the distal portion of a net or mesh can be less than 0.025 N/mm. In another example, the Young's modulus of (the material used to make) the distal portion of a net or mesh can be between 0.001 and 0.005. Alternatively, the flexibility (or elasticity, stretchability, pliability, and/or softness) of a distal portion of a net or mesh relative to that of a proximal portion of the net or mesh can be expressed as a percentage. In another example, the stiffness of the proximal portion of a net or mesh can be greater than 0.025 N/mm.

In an example, an opening through a flexible net or mesh through which embolic members and/or embolic material is inserted can be centrally located with respect to the proximal portion of the net or mesh. In an example, an opening through a flexible net or mesh through which embolic members and/or embolic material is inserted can be aligned with the longitudinal axis of the proximal portion of the net or mesh. In an example, an opening through a flexible net or mesh through which embolic members and/or embolic material is inserted can be connected to a catheter. In an example, an opening through a flexible net or mesh through which embolic members and/or embolic material is inserted can be detachably connected to a catheter. In an example, an opening through a flexible net or mesh through which embolic members and/or embolic material is inserted can be connected to a catheter, wherein this connection can be broken by application of electromagnetic energy.

In an example, an opening through a flexible net or mesh through which embolic members and/or embolic material is inserted can be formed by an annular member. In an example, an opening through a flexible net or mesh through which embolic members and/or embolic material is inserted can be formed by an annular member selected from the group consisting of a ring, band, cylinder, tube, or catheter. In an example, an opening through a flexible net or mesh through which embolic members and/or embolic material is inserted can be formed by one or more rings, bands, cylinders, tubes, or catheters. In an example, an opening through a flexible net or mesh through which embolic members and/or embolic material is inserted can be formed by two or more nested (e.g. concentric) rings, bands, cylinders, tubes, or catheters. In an example, annular members which form an opening through a flexible net or mesh can be rings or bands which encircle the ends of the net or mesh.

In an example, an annular member which forms an opening through a flexible net or mesh can be a metal ring, band, or cylinder. In an example, an annular member which forms an opening through a flexible net or mesh can be a polymer ring, band, or cylinder. In an example, an annular member which forms an opening through a flexible net or mesh can be a wire, cord, or string. In an example, an annular member which forms an opening through a flexible net or mesh can be a ring or band which encircles a net or mesh, thereby radially-constraining and/or pinching a net or mesh but allowing embolic members and/or embolic material to pass through it into the interior and/or a concavity of the flexible net or mesh. In an example, an annular member which forms an opening through a flexible net or mesh can be a cylinder which encircles a net or mesh, thereby radially-constraining and/or pinching a net or mesh but allowing embolic members and/or embolic material to pass through it into the interior and/or a concavity of the flexible net or mesh.

In an example, an annular member which forms an opening through a flexible net or mesh can be a cord or wire which encircles a net or mesh, thereby radially-constraining and/or pinching a net or mesh but allowing embolic members and/or embolic material to pass through it into the interior and/or a concavity of the flexible net or mesh. In an example, an annular member which forms an opening through a flexible net or mesh can be a catheter or tube around which a net or mesh is attached, thereby radially-constraining and/or pinching a net or mesh but allowing embolic members and/or embolic material to pass through it into the interior and/or a concavity of the flexible net or mesh. In an example, an annular member which forms an opening through a flexible net or mesh can be a lumen through a flexible net or mesh through which embolic members and/or material is inserted into the flexible net or mesh.

In an example, a net or mesh can be soldered, melted, glued, or crimped onto an annular member which forms an opening through a flexible net or mesh. In an example, an annular member which forms an opening through a flexible net or mesh can have an inner ring and an outer ring, wherein a net or mesh is fixed (e.g. soldered, melted, glued, or crimped) between the two rings. In an example, an annular member which forms an opening through a flexible net or mesh can comprise an inner ring or cylinder and an outer elastic band, wherein a net or mesh is held between the inner and outer portions. In an example, an annular member which forms an opening through a flexible net or mesh can be centrally-located with respect to a proximal surface of the flexible net or mesh. In an example, an annular member which forms an opening through a flexible net or mesh can be centrally-located with respect to the longitudinal axis of the flexible net or mesh. In an example, an annular member which forms an opening through a flexible net or mesh can be a hub into which proximal ends of braided wires or tubes of the stent are bound or attached. In an example, an annular member which forms an opening through a flexible net or mesh can be off-axial with respect to the longitudinal axis of the flexible net or mesh.

In an example, an annular member which forms an opening through a flexible net or mesh can comprise two nested and/or concentric (inner and outer) cylinders, wherein a net or mesh is pinched and/or crimped between the two cylinders. In an example, an annular member which forms an opening through a flexible net or mesh can comprise two nested and/or concentric (inner and outer) rings or bands, wherein a net or mesh is pinched and/or crimped between the two rings or bands. In an example, an annular member which forms an opening through a flexible net or mesh can comprise two nested and/or concentric (inner and outer) cylinders, wherein a net or mesh is melted or glued between the two cylinders. In an example, an annular member which forms an opening through a flexible net or mesh can comprise two nested and/or concentric (inner and outer) rings or bands, wherein a net or mesh is melted or glued between the two rings or bands.

In an example, an annular member which forms an opening through a flexible net or mesh can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member which forms an opening through a flexible net or mesh can be attached to a catheter during delivery of embolic members and/or material, and then detached (e.g. by the application of electromagnetic energy) from the catheter after delivery of the embolic members and/or material.

In an example, an annular member which forms an opening through a flexible net or mesh can have an outer diameter which is between 5% and 20% of the diameter of a net or mesh before a net or mesh is radially constrained. In an example, an annular member which forms an opening through a flexible net or mesh can have an outer diameter which is between 10% and 33% of the diameter of a net or mesh before a net or mesh is radially constrained. In an example, an annular member which forms an opening through a flexible net or mesh can have an outer ring (or cylinder) with a first diameter and an inner ring (or cylinder) with a second diameter, wherein a net or mesh is crimped or pinched between the outer ring (or cylinder) and inner ring (or cylinder), and wherein the first diameter is between 50% and 75% of the second diameter. In an example, an annular member which forms an opening through a flexible net or mesh can have an outer ring (or cylinder) with a first diameter and an inner ring (or cylinder) with a second diameter, wherein a net or mesh is crimped or pinched between the outer ring (or cylinder) and inner ring (or cylinder), and wherein the first diameter is between 66% and 90% of the second diameter.

In an example, an annular member which forms an opening through a flexible net or mesh can comprise two nested rings, bands, or cylinders, wherein a section of a net or mesh is inserted and held between the nested rings, bands, or cylinders. In an example, an annular member which forms an opening through a flexible net or mesh can comprise an outer ring, band, or cylinder and an inner ring, band, or cylinder, wherein a section of a net or mesh is inserted and held between them. In an example, an annular member which forms an opening through a flexible net or mesh can comprise an outer ring, band, or cylinder and an inner ring, band, or cylinder, wherein one or both of the rings, bands, or cylinders are threaded. In an example, an annular member which forms an opening through a flexible net or mesh can comprise an outer ring, band, or cylinder and an inner ring, band, or cylinder, wherein one or both of the rings, bands, or cylinders has a helical thread. In an example, an annular member which forms an opening through a flexible net or mesh can comprise an outer ring, band, or cylinder and an inner ring, band, or cylinder, wherein one or both of the rings, bands, or cylinders has a helical thread to hold a section of a net or mesh.

In an example, this device can further comprise a closure mechanism which closes an opening through an annular member. In an example, this closure mechanism can be closed by the operator of the device after embolic members and/or material has been inserted into a flexible net or mesh. In an example, this closure mechanism can require action by a user during the procedure to close off the opening. In various examples, this closure mechanism can comprise a drawstring, loop, seal, fusible member, adhesive, snap, clip, valve, or cap. In an example, this closure mechanism can close automatically after embolic members and/or material has been inserted into a flexible net or mesh. In an example, a closure mechanism can be a valve. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a closure mechanism which closes an opening through an annular member can be an electric detachment mechanism. In an example, this closure mechanism can be an elastic ring or band. In an example, this closure mechanism can be a threaded mechanism. In an example, this closure mechanism can be a sliding cover. In an example, this closure mechanism can be a sliding plug. In an example, this closure mechanism can be a filament loop. In an example, this closure mechanism can be an electromagnetic solenoid.

In an example, a closure mechanism which closes an opening through a flexible net or mesh can comprise a leaflet valve. In an example, this leaflet valve can be positioned in an opening (or lumen) through a mesh (or net) which bridges an aneurysm neck. When this leaflet valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this leaflet valve is in its closed configuration, it reduces blood flood through the opening in the mesh into the aneurysm. In other words, this leaflet valve can serve as a “closure mechanism” for an intrasacular aneurysm occlusion device.

In an example, a closure mechanism which closes an opening through a flexible net or mesh can comprise a bi-leaflet valve or a tri-leaflet valve, analogous to a heart valve. In an example, a valve can passively open when an embolic member is pushed through it and can passively close after the member passes through or when a portion of the member is detached. In an example, such a valve can allow an embolic member to be inserted into the flexible net, but the valve closes to reduce blood flow after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire.

In an example, a leaflet valve can have a single leaflet or flap. In an example, a leaflet valve can have four or more leaflets or flaps. In an example, a leaflet valve can passively open when an embolic member (such as an embolic coil, hydrogel, microsponge, bead, or a string-of-pearls embolic strand) pushes through it. In an example, a leaflet valve can passively close when after the embolic member has passed through. In an example, a leaflet valve can be made from an elastomeric material. In an example, a leaflet valve can be made from a silicone-based polymer. In an example, a leaflet valve can be made from rigid material such as metal. In an example, a leaflet valve can be made from titanium and carbon. In an example, a leaflet valve can be remotely opened and/or closed by the operator of the device. In an example, a leaflet valve can be remotely opened and/or closed by an operator by the application of electromagnetic energy.

In an example, a closure mechanism which closes an opening through a flexible net or mesh can comprise an elastic annular valve. In an example, this elastic annular valve can be positioned in an opening (or lumen) through a mesh (or net) which bridges an aneurysm neck. When this elastic annular valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this elastic annular valve is in its closed configuration, it reduces blood flood through the opening in the mesh into the aneurysm. In other words, this elastic annular valve can serve as a “closure mechanism” for an intrasacular aneurysm occlusion device. We all have times when we need closure. In an example, an elastic annular valve can passively open when an embolic member (such as an embolic coil, hydrogel, microsponge, bead, or a string-of-pearls embolic strand) pushes through it. In an example, an elastic annular valve can passively close when after the embolic member has passed through. In an example, an elastic annular valve can be made from an elastomeric material. In an example, an elastic annular valve can be made from a silicone-based polymer.

In an example, a closure mechanism which closes an opening through a flexible net or mesh can comprise a rotational valve. A rotational valve can comprise an (outer) first layer with a first opening (or hole) and an (inner) second layer with a second opening (or hole). When the first and second openings (holes) are not aligned, then the valve is in its closed configuration. When the first and second openings (holes) are aligned, then the valve is in its open configuration. In this example, the valve is changed from its closed configuration to its open configuration, or vice versa, by rotating (or revolving, pivoting, turning, or twisting) the first layer relative to the second layer, or vice versa. In an example, a rotational valve can comprise two or more overlapping (e.g. parallel) layers with openings (holes). When the openings (holes) of different layers are not aligned, then the valve is closed. When the opening (holes) of different layers are aligned, then the valve is open. In an example, the valve can be opened or closed by rotating one layer relative to the other layer. In an example, one or both layers can be rotated remotely by the operator of the device, enabling the operator to open or close the valve remotely.

In an example, a closure mechanism which closes an opening through a flexible net or mesh can comprise a rotational valve which is positioned in an opening (or lumen) through a mesh (or net) which bridges an aneurysm neck. When this rotational valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this rotational valve is in its closed configuration, it reduces blood flood through the opening in the mesh into the aneurysm. In other words, this rotational valve can serve as a “closure mechanism” for an intrasacular aneurysm occlusion device.

In an example, a closure mechanism which closes an opening through a flexible net or mesh can comprise a sliding valve. In an example, a sliding valve can comprise a layer with an opening (or hole) and a sliding flap (or lid). When the sliding flap (lid) covers the opening (hole), then the valve is in its closed configuration. When the sliding flap (lid) does not cover the opening (hole), then the valve is in its open configuration. In this example, the valve is changed from its closed configuration to its open configuration, or vice versa, by moving the sliding flap. In an example, the sliding flap can be moved remotely by the operator of the device, enabling the operator to open or close the valve remotely.

In an example, a closure mechanism which closes an opening through a flexible net or mesh can comprise a sliding valve which is positioned in an opening (or lumen) through a mesh (or net) which bridges an aneurysm neck. When this sliding valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this sliding valve is in its closed configuration, it reduces blood flood through the opening in the mesh into the aneurysm. In other words, this sliding valve can serve as a “closure mechanism” for an intrasacular aneurysm occlusion device.

In an example, a closure mechanism which closes an opening through a flexible net or mesh can comprise a pivoting valve. A pivoting value can comprise a lumen (opening) with a pivoting flap (or plug). When the pivoting flap (plug) blocks the lumen (opening), then the valve is in its closed configuration. When the pivoting flap (lid) does not block the lumen (opening), then the valve is in its open configuration. In this example, the valve is changed from its closed configuration to its open configuration, or vice versa, by pivoting (rotating) the flap around a central axis. In the example of a square opening, a valve could changed from its closed configuration to its open configuration, or vice versa, by pivoting (rotating) a flap around one side. In an example, the pivoting flap can be moved remotely by the operator of the device, enabling the operator to open or close the valve remotely. This type of pivoting valve is analogous to the valves which are used in circular air ducts for HVAC (heating, ventilation, and air conditioning) systems in buildings.

In an example, a closure mechanism which closes an opening through a flexible net or mesh can comprise a pivoting valve which is positioned in an opening (or lumen) through a mesh (or net) which bridges an aneurysm neck. When this pivoting valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. This type of pivoting valve is more appropriate for liquid embolic material than for coils, beads, or string-of-pearls strands which might get snagged on it. When this pivoting valve is in its closed configuration, it reduces blood flood through the opening in the mesh into the aneurysm. In other words, this pivoting valve can serve as a “closure mechanism” for an intrasacular aneurysm occlusion device. We all have times when we need closure.

In an example, a closure mechanism which closes an opening through a flexible net or mesh can comprise a plug mechanism. In an example, an active valve can be remotely opened and/or closed by an operator by cutting, pulling, or pushing a flap or plug. In an example, a plug mechanism can comprise a lumen (opening) and a plug which is inserted into the lumen. When a plug blocks the lumen (opening), then the plug mechanism is in its closed configuration. When a plug does not block the lumen (opening), then the plug mechanism is in its open configuration. In this example, the plug mechanism is changed from its open configuration to its closed configuration by inserting a plug into the lumen (opening). In an example, a plug can be inserted remotely by the operator of the device, enabling the operator to close the plug mechanism remotely. In an example, a plug can be inserted into a lumen by using a guidewire or hydraulic pressure. In an example, a plug can be made from hydrogel.

In an example, a closure mechanism which closes an opening through a flexible net or mesh can comprise a plug mechanism which is positioned in an opening (or lumen) through a mesh (or net) which bridges an aneurysm neck. When this plug mechanism is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this plug mechanism is in its closed configuration, it reduces blood flood through the opening in the mesh into the aneurysm.

In an example, insertion of embolic members and/or material into a flexible net or mesh causes the flexible net or mesh to expand and conform to the walls of even an irregularly-shaped aneurysm sac. In an example, a flexible net or mesh can self-expand to a first extent after being released from a catheter into an aneurysm sac and net or mesh can further expand, to a second extent, due to pressure from the accumulation of embolic members and/or embolic material inside the net or mesh. In an example, a flexible net or mesh can further expand to conform to the wall contours of even an irregularly-shaped aneurysm sac.

In an example, embolic members and/or material which are inserted into a flexible net or mesh in an aneurysm sac can comprise one or more longitudinal metal coils. In an example, embolic members and/or material can comprise one or more longitudinal mesh ribbons. In an example, embolic members and/or material can comprise one or more longitudinal polymer strands. In an example, embolic members and/or material can comprise one or more string-of-pearls embolic strands, wherein a string-of-pearls embolic strand is a plurality of embolic beads or other embolic masses connected by a longitudinal wire, filament, string, cord, yarn, or thread. In an example, embolic members and/or material can comprise a plurality of hydrogel pieces or microsponges. In an example, embolic members and/or material can comprise liquid or gel which congeals after delivery into the flexible net or mesh.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from tantalum. In an example, embolic members can be made from cobalt-chrome alloy (cobalt chromium).

In an example, embolic members can be made from polyetherether ketone (PEEK). In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with embolic members and stop them from sliding, effectively locking them in proximity to each other.

In an example, embolic members can be conveyed through a lumen to an aneurysm in a fluid flow, wherein the fluid escapes out from a net or mesh and embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a method to occlude a cerebral aneurysm can comprise: receiving a 3D image of a cerebral aneurysm; and analyzing the 3D image to estimate an optimal amount of embolic members or material to be inserted into the aneurysm in order to occlude the aneurysm. In an example, an optimal amount of embolic members or material can be calculated by estimating the total interior volume of the aneurysm based on the 3D image of the aneurysm. In an example, estimation of the optimal amount of embolic members or material can depend on one or more factors selected from the group consisting of: general aneurysm shape or type; aneurysm size; aneurysm location; aneurysm rupture status; type of embolic material; shape of embolic material; size of embolic material; softness and/or compressibility of embolic material; parent vessel shape; parent vessel location; patient demographic information; and patient medical history.

In an example, embolic members or material can comprise embolic coils or ribbons. In an example, embolic members or material can comprise hydrogels or other gelatinous material. In an example, embolic members or material can comprise a string-of-pearls structure (i.e. a plurality of embolic members connected by a string, filament, wire, or micro-chain). In an example, a flow of liquid or gelatinous embolic material can be (automatically) pumped into a cerebral aneurysm until the optimal amount of embolic material has been dispensed. In an example, embolic members or material can be (automatically) delivered into a cerebral aneurysm until the optimal amount of embolic members has been dispensed.

In an example, this invention can be embodied in a method to create a device to occlude a cerebral aneurysm comprising: receiving a 3D image of a cerebral aneurysm; creating a 3D model or 3D mandrel based on the 3D image; and wrapping, weaving, braiding, melting, shrinking, or otherwise conforming wires around the 3D model or 3D mandrel in order to create a custom-shaped convex flexible wire mesh which is configured to be inserted into the aneurysm. In an example, embolic members or material can be inserted into the custom-shaped convex flexible wire mesh after the mesh has been inserted into the cerebral aneurysm.

In an example, an in-vivo 3D image of a cerebral aneurysm can be created by a medical imaging method selected from the group consisting of: Computerized Tomography (CT), Computerized Tomography Angiography (CTA), Cone Beam Computed Tomography (CBCT), Conoscopic Holography (CH), Digital Subtraction Angiography (DSA), Magnetic Resonance Angiography (MRA), Magnetic Resonance Imaging (MRI), Maximum Intensity Projection (MIP), Medical Holographic Imaging (MHI), Micro Computerized Tomography (MCT), Positron Emission Tomography (PET), Tuned-Aperture Computed Tomography (TACT), Doubting Thomagraphy (DT), and Ultrasound (U/S). In an example, an in-vivo 3D image of a cerebral aneurysm can be a digital image. In an example, an in-vivo 3D image of a cerebral aneurysm can be a volumetric image. In an example, an in-vivo 3D image can be constructed by digitally merging a plurality of 2D images from different perspectives or at different times. In an example, an in-vivo 3D image of a cerebral aneurysm can be created after injection of contrast media into a person's bloodstream.

In an example, there can be an optimal amount (or an optimal range of amounts) of embolic members or material which should be inserted into a flexible net, mesh, bag, liner, or stent within an aneurysm with a particular size and shape in order to occlude that aneurysm most effectively and safely. If the amount of embolic members or material inserted into a flexible net, mesh, bag, liner, or stent is less than this optimal amount, then there may be gaps between the flexible net, mesh, bag, liner, or stent and the walls of the aneurysm which allow blood to continue to flow into the aneurysm. If the amount of embolic members or material inserted into a flexible net, mesh, bag, liner, or stent is greater than this optimal amount, then the flexible net, mesh, bag, liner, or stent may exert too much pressure on the aneurysm walls (potentially causing the aneurysm to rupture); the flexible net, mesh, bag, liner, or stent may protrude out of the aneurysm into the parent vessel; or the flexible net, mesh, bag, liner, or stent may leak embolic material.

An optimal amount (or an optimal range of amounts) of embolic members or material to be inserted into a flexible net, mesh, bag, liner, or stent can be estimated by human judgment. In an example, estimation by human judgment of an optimal amount of embolic members or material to be inserted into a flexible net, mesh, bag, liner, or stent can be done based on medical imaging before an aneurysm occlusion procedure. In an example, estimation by human judgment of an optimal amount of embolic members or material to be inserted into a flexible net, mesh, bag, liner, or stent can be done based on real-time medical imaging during an aneurysm occlusion procedure.

However, an automated process to estimate an optimal amount of embolic members or material, such as a process using computer analysis of digital 3D images of an aneurysm, can be more accurate and quicker than estimation based on human judgment. This can help to reduce errors of under or over injection of embolic members or material and can also help to reduce aneurysm occlusion procedure time. In an example, there can be automated estimation of an optimal amount (or optimal amount range) of embolic members or material to be inserted into a flexible net, mesh, bag, liner, or stent in an cerebral aneurysm based on analysis of 3D images of that aneurysm.

In an example, an optimal amount (or an optimal range of amounts) of embolic members or material can be expressed as a volume, especially for a liquid or gelatinous embolic material which is dispensed (into a flexible net, mesh, bag, liner, or stent) in a flow. In an example, an optical amount (or an optimal range of amounts) of embolic members or material can be expressed as a percentage the interior volume of an aneurysm. In an example, an optimal amount (or optimal amount range) of embolic material volume can be calculated in steps comprising: (a) estimating the total interior volume of an aneurysm based on 3D images of the aneurysm; (a) subtracting the volume of the perimeter layer of a flexible net, mesh, bag, liner, or stent which is inserted into the aneurysm in order to calculate a remaining interior volume; and (c) expressing the optimal volume of embolic material to be inserted into the flexible net, mesh, bag, liner, or stent as a percentage of the remaining interior volume.

In an example, a string-of-pearls embolic structure which is inserted into a flexible net, mesh, bag, liner, or stent can comprise a plurality of embolic members (e.g. microsponges, microspheres, beads, or hydrogels) which are connected by one or more longitudinal flexible members (e.g. filaments, strings, threads, fibers, sutures, yarns, coils, or wires), wherein surfaces of the embolic members have microscale (or nanoscale) hook-and-eye structures which cause the embolic members to stick to each other upon contact. This can help to prevent the embolic members from leaking out of the flexible net, mesh, bag, liner, or stent. In an example, the embolic members can be separated from each other during delivery through a lumen so that they do not bunch together and clog the lumen, but can come into engaging contact with each other once they exit the lumen into the flexible net, mesh, bag, liner, or stent.

In an example, embolic members can adhere to each other. In an example, embolic members can adhere to each other after they are inserted into a flexible net, mesh, bag, liner, or stent so that they are less likely to escape out of holes in the flexible net, mesh, bag, liner, or stent. In an example, embolic members can adhere to each other after they are inserted into an aneurysm so that they are less likely to protrude out of the aneurysm into the parent vessel. In an example, embolic members or material can have a first level of adhesion (or stickiness) before they are inserted into an aneurysm and have a second level of adhesion (or stickiness) after they are inserted into the aneurysm, wherein the second level is greater than the first level. In an example, embolic members or material can be changed from a first level of adhesion (or stickiness) to a second level of adhesion (or stickiness) by a means selected from the group consisting of: exposure to blood; exposure to body thermal energy; selective application of a chemical substance by a provider and/or device operator; selective application of electromagnetic energy by a provider and/or device operator; selective application of light energy in a selected wavelength by a provider and/or device operator; and selective application of thermal energy by a provider and/or device operator.

In an example, embolic members or material can be fused, congealed, stuck, or adhered together after insertion into an aneurysm by selective intrasacular application of a chemical substance by a provider and/or device operator. In an example, embolic members or material can be fused, congealed, stuck, or adhered together after insertion by selective intrasacular application of electromagnetic energy by a provider and/or device operator. In an example, embolic members or material can be fused, congealed, stuck, or adhered together after insertion by selective intrasacular application of light energy in a selected wavelength by a provider and/or device operator. In an example, embolic members or material can be fused, congealed, stuck, or adhered together after insertion by selective intrasacular application of thermal energy by a provider and/or device operator.

In an example, the delivery of an optimal amount of embolic material or an optimal number of embolic members can be partially or fully automated based on an estimated amount (or range of amounts). In an example, a device can automatically control the amount of embolic material and/or number of embolic members inserted into a flexible net, mesh, bag, liner, or stent in order to insert an optimal amount of embolic material or an optimal number of embolic members. In an example, a device can automatically pump a flow of liquid or gelatinous embolic material into a flexible net, mesh, bag, liner, or stent until the optimal amount of embolic material has been dispensed. In an example, a device can automatically push a series of longitudinal embolic members (such as coils) into a flexible net, mesh, bag, liner, or stent until the optimal amount or number of embolic members has been dispensed. In an example, a device can automatically deliver a plurality of embolic members into a flexible net, mesh, bag, liner, or stent until the optimal number of embolic members has been dispensed.

In an example, a device can further comprise an embolic delivery component which measures and controls the insertion of embolic members or material into a flexible net, mesh, bag, liner, or stent within an aneurysm so that the optimal amount of embolic members or material is inserted. In an example, an embolic delivery component for a longitudinal embolic member or series of longitudinal members (such as coils) can measure and control the length or number of embolic members inserted into a flexible net, mesh, bag, liner, or stent within an aneurysm. In an example, an embolic delivery component to deliver an optimal length or number of longitudinal embolic members into a flexible net, mesh, bag, liner, or stent within an aneurysm can push a desired length or number of embolic members through a lumen into a flexible net, mesh, bag, liner, or stent within an aneurysm.

In an example, an embolic delivery component can selectively cut, sever, snap, melt, or segment (and detach) an otherwise continuous length of embolic material (such as a coil) after the optimal length of the material has been inserted into a flexible net, mesh, bag, liner, or stent within an aneurysm. In an example, an embolic delivery component can cut a longitudinal embolic member and detach the severed portion after an optimal length of the longitudinal embolic member has been inserted into a flexible net, mesh, bag, liner, or stent within an aneurysm. In an example, an embolic delivery component can melt a longitudinal embolic member and detach the severed portion after an optimal length of the longitudinal embolic member has been inserted into a flexible net, mesh, bag, liner, or stent within an aneurysm.

In an example, an embolic delivery component for a liquid or gelatinous embolic material can measure and control the flow of this embolic material into a flexible net, mesh, bag, liner, or stent within an aneurysm. In an example, an embolic delivery component for delivering a liquid or gelatinous embolic material can comprise a pump. In an example, an embolic delivery component for delivering a liquid or gelatinous embolic material can be selected from the group consisting of: axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, In various examples, infusion pump, mechanical screw-type pump, Micro Electrical Mechanical System (MEMS) pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump.

In an example, a liquid or gelatinous embolic material inserted into a flexible net, mesh, bag, liner, or stent can be a liquid polymer. In an example, a liquid or gelatinous embolic material inserted into a flexible net, mesh, bag, liner, or stent can be selected from the group consisting of: 2-octyl cyanoacrylate; ethyl-2-cyanoacrylate; methyl 2-cyanoacrylate; and n-butyl cyanoacrylate. In an example, a liquid or gelatinous embolic material inserted into a flexible net, mesh, bag, liner, or stent can be selected from the group consisting of: acrylamide-based hydrogel; acrylic-acid-based hydrogel; agar; alginate-based hydrogel; carboxymethyl cellulose; cellulose; chitin; chitosan; collagen; copolymeric hydrogel; gellan; gum arabic; heparin; homopolymeric hydrogel; hyaluronan; hydrocolloid hydrogel; methyl cellulose; multipolymer interpenetrating polymeric hydrogel; pectin; pluronic-acid-based hydrogel; polyacrylic-acid-based hydrogel; polypeptide-based; polyurethane-based; poly-vinyl-alcohol-based hydrogel; starch; superabsorbent hydrogel; superporous hydrogel; and xanthan

In an example, guidance concerning the optimal amount of embolic members or material can be partially, but not fully, automated. In an example, an embolic delivery component can track and display the cumulative amount of embolic members or material which is being inserted into a flexible net, mesh, bag, liner, or stent during a procedure. In an example, an embolic delivery component can notify a provider and/or device operator in real time (e.g. with a visual, auditory, or tactile signal) as the cumulative amount of inserted embolic members or material is approaching the optimal amount. In an example, an embolic delivery component can notify a provider and/or device operator in real time (e.g. with a visual, auditory, or tactile signal) as the cumulative amount of inserted embolic members or material has reached the optimal amount. Example variations discussed in other portions of this disclosure or in priority-linked disclosures can also be applied to these examples where relevant, but are not repeated here in order to reduce narrative redundancy.

In an example, selected types of embolic members or material (e.g. those which are less likely to protrude out of an aneurysm into a parent vessel) can be delivered directly into an aneurysm sac without the need for a flexible net, mesh, bag, liner, or stent. In such examples, it can be useful to have an automated method for estimating the optimal amount of embolic members or material to be inserted into the aneurysm based on 3D imaging of the aneurysm. In an example, a method to determine an optimal amount of embolic members or material to be inserted into an cerebral aneurysm can comprise: (a) receiving a 3D image of a cerebral aneurysm; and (b) analyzing the 3D image to estimate an optimal amount (or an optimal range of amounts) of embolic members or material to be inserted into the aneurysm in order to occlude the aneurysm.

In an example, embolic members or material inserted into an aneurysm can be biocompatible yarn or fabric. In an example, embolic members or material inserted into an aneurysm can be blobs of gel. In an example, embolic members or material inserted into an aneurysm can be embolic coils. In an example, embolic members or material inserted into an aneurysm can be embolic gel which solidifies after insertion. In an example, embolic members or material inserted into an aneurysm can be embolic glue. In an example, embolic members or material inserted into an aneurysm can be embolic liquid which solidifies after insertion.

In an example, embolic members or material inserted into an aneurysm can be fiber strips. In an example, embolic members or material inserted into an aneurysm can be flexible wires. In an example, embolic members or material inserted into an aneurysm can be hydrogels. In an example, embolic members or material inserted into an aneurysm can be mesh ribbon. In an example, embolic members or material inserted into an aneurysm can be micro-beads. In an example, embolic members or material inserted into an aneurysm can be microscale mesh spheres.

In an example, embolic members or material inserted into an aneurysm can be microspheres. In an example, embolic members or material inserted into an aneurysm can be microsponges. In an example, embolic members or material inserted into an aneurysm can be stream of paste which solidifies after insertion. In an example, embolic material can comprise a shredded musical score, wherein a person can have a catchy tune stuck in their head. In an example, embolic members or material can be selected from the group consisting of: biocompatible yarn or fabric; blobs of gel; embolic coils; embolic gel which solidifies after insertion; embolic glue; embolic liquid which solidifies after insertion; fiber strips; flexible wires; hydrogels; mesh ribbon; micro-beads; microscale mesh spheres; microspheres; microsponges; stream of paste which solidifies after insertion; and string-of-pearls embolic structure (e.g. a plurality of embolic members connected by a string, filament, wire, or micro-chain). In an example, embolic members or material inserted into an aneurysm can be a string-of-pearls embolic structure (e.g. a plurality of embolic members inserted into an aneurysm connected by a string, filament, wire, or micro-chain).

In an example, embolic members can adhere to each other. In an example, embolic members can adhere to each other after they are inserted into a flexible net, mesh, bag, liner, or stent so that they are less likely to escape out of holes in the flexible net, mesh, bag, liner, or stent. In an example, embolic members can adhere to each other after they are inserted into an aneurysm so that they are less likely to protrude out of the aneurysm into the parent vessel. In an example, embolic members or material can have a first level of adhesion (or stickiness) before they are inserted into an aneurysm and have a second level of adhesion (or stickiness) after they are inserted into the aneurysm, wherein the second level is greater than the first level. In an example, embolic members or material can be changed from a first level of adhesion (or stickiness) to a second level of adhesion (or stickiness) by a means selected from the group consisting of: exposure to blood; exposure to body thermal energy; selective application of a chemical substance by a provider and/or device operator; selective application of electromagnetic energy by a provider and/or device operator; selective application of light energy in a selected wavelength by a provider and/or device operator; and selective application of thermal energy by a provider and/or device operator.

In an example, embolic members or material can be fused, congealed, stuck, or adhered together after insertion by selective intrasacular application of a chemical substance by a provider and/or device operator. In an example, embolic members or material can be fused, congealed, stuck, or adhered together after insertion by selective intrasacular application of electromagnetic energy by a provider and/or device operator. In an example, embolic members or material can be fused, congealed, stuck, or adhered together after insertion by selective intrasacular application of light energy in a selected wavelength by a provider and/or device operator. In an example, embolic members or material can be fused, congealed, stuck, or adhered together after insertion by selective intrasacular application of thermal energy by a provider and/or device operator.

In an example, a string-of-pearls embolic structure which is inserted into an aneurysm can comprise a plurality of embolic members (e.g. microsponges, microspheres, beads, or hydrogels) which are connected by one or more longitudinal flexible members (e.g. filaments, strings, threads, fibers, sutures, yarns, coils, or wires), wherein the surfaces of the embolic members have microscale (or nanoscale) hook-and-eye structures which cause the embolic members to stick to each other upon contact. This can help to prevent the embolic members from protruding out of the aneurysm. In an example, the embolic members can be separated from each other during delivery through a lumen so that they do not bunch together and clog the lumen, but can come into engaging contact with each other once they exit the lumen into the aneurysm.

In an example, an embolic delivery component for a liquid or gelatinous embolic material can measure and control the flow of this embolic material into an aneurysm. In an example, an embolic delivery component for a liquid or gelatinous embolic material can comprise a pump. In an example, an embolic delivery component for a liquid or gelatinous embolic material can be selected from the group consisting of: axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, In various examples, infusion pump, mechanical screw-type pump, Micro Electrical Mechanical System (MEMS) pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump.

In an example, a liquid or gelatinous embolic material can be selected from the group consisting of: 2-octyl cyanoacrylate; ethyl-2-cyanoacrylate; methyl 2-cyanoacrylate; and n-butyl cyanoacrylate. In an example, a liquid or gelatinous embolic material can be selected from the group consisting of: acrylamide-based hydrogel; acrylic-acid-based hydrogel; agar; alginate-based hydrogel; carboxymethyl cellulose; cellulose; chitin; chitosan; collagen; copolymeric hydrogel; gellan; gum arabic; heparin; homopolymeric hydrogel; hyaluronan; hydrocolloid hydrogel; methyl cellulose; multipolymer interpenetrating polymeric hydrogel; pectin; pluronic-acid-based hydrogel; polyacrylic-acid-based hydrogel; polypeptide-based; polyurethane-based; poly-vinyl-alcohol-based hydrogel; starch; superabsorbent hydrogel; superporous hydrogel; and xanthan. Other example variations discussed elsewhere in this disclosure or in priority-linked disclosures can also be applied to an example where relevant.

I claim:

1. An intrasacular aneurysm occlusion device comprising:
a flexible net or mesh which is inserted into and expanded within an aneurysm sac;
wherein the flexible net or mesh further comprises a proximal portion whose centroid is a first distance from the aneurysm neck after expansion within the aneurysm sac; and wherein the proximal portion has a first average level of flexibility and a first average level of stiffness;
wherein the flexible net or mesh further comprises a distal portion whose centroid is a second distance from the aneurysm neck after expansion within the aneurysm sac; wherein the distal portion has a second average level of flexibility and a second average level of stiffness; wherein the first distance is less than the second distance; wherein the first average level of flexibility is less than the second average level of flexibility and/or the first average level of stiffness is greater than the second average level of stiffness;
an opening through the proximal portion of the flexible net or mesh;
embolic members and/or embolic material which is inserted through the opening into the flexible net or mesh, wherein insertion of the embolic members and/or material into the flexible net or mesh causes the flexible net or mesh to expand and conform to the walls of even an irregularly-shaped aneurysm sac; and
a closure mechanism which closes the opening after embolic members and/or material has been inserted through the opening into the flexible net or mesh.

2. The device in claim 1 wherein elasticity, stretchability, conformability, pliability, or softness is substituted for flexibility as a measured characteristic of the proximal and distal portions of the net or mesh.

3. The device in claim 1 wherein Young's Modulus, resiliency, strength, or durometer is substituted for stiffness as a measured characteristic of the proximal and distal portions of the net or mesh.

4. The device in claim 1 wherein the stiffness of the proximal portion of the net or mesh relative to that of the distal portion of the net or mesh is increased by using thicker wires, tubes, filaments, and/or strands for the proximal portion than those used for the distal portion.

5. The device in claim 1 wherein the stiffness of the proximal portion of the net or mesh relative to that of the distal portion of the net or mesh is increased by using stiffer wires, tubes, filaments, and/or strands for the proximal portion than those used for the distal portion.

6. The device in claim 1 wherein the stiffness of the proximal portion of the net or mesh relative to that of the distal portion of the net or mesh is increased by creating a greater density of wires, tubes, filaments, and/or strands in the proximal portion than in the distal portion.

7. The device in claim 1 wherein the stiffness of the proximal portion of the net or mesh relative to that of the distal portion of the net or mesh is increased by using a first proportion of metal relative to polymer to create the proximal portion and using a second proportion of metal relative to polymer to create the distal portion, wherein the second proportion is less than the first proportion.

8. The device in claim 1 wherein the stiffness of the proximal portion of the net or mesh relative to that of the distal portion of the net or mesh is increased by adding radial spokes or struts to the proximal portion, wherein a radial array of wires, tubes, or struts extend radially-outward from a central area of the proximal portion of the net or mesh.

9. The device in claim 1 wherein the stiffness of the proximal portion of the net or mesh relative to that of the distal portion is increased by integrating an array of nested wire rings into the proximal portion.

10. The device in claim 1 wherein the stiffness of the proximal portion of the net or mesh relative to that of the distal portion is increased by integrating an undulating ring of wire into the proximal portion.

11. The device in claim 1 wherein the stiffness of the proximal portion of the net or mesh relative to that of the distal portion is increased by integrating a helical wire into the proximal portion of the net or mesh.

12. The device in claim 1 wherein the stiffness of the proximal portion of the net or mesh relative to that of the distal portion is increased by integrating one or more coils into the proximal portion of the net or mesh.

13. The device in claim 1 wherein the stiffness of the proximal portion of the net or mesh relative to that of the distal portion is increased by coating wires, tubes, filaments, and/or strands in the proximal portion of the net or mesh with a stiffening material.

14. The device in claim 1 wherein the stiffness of the proximal portion of the net or mesh relative to that of the distal portion is increased by using stiffer material, such as material with a higher Young's Modulus and/or durometer, to create the proximal portion than to create the distal portion.

15. The device in claim 1 wherein the stiffness of the proximal portion of the net or mesh relative to that of the distal portion of the net or mesh is increased by having a greater number of layers in the proximal portion than in the distal portion.

16. The device in claim 1 wherein the flexible net or mesh comprises a convex spherical, ellipsoidal, and/or generally-globular mesh at least partially within the concavity of a proximal concave mesh with a distal-facing concavity.

17. The device in claim 1 wherein the flexible net or mesh comprises a convex spherical, ellipsoidal, and/or generally-globular mesh made primarily or entirely from a polymer and at least partially within the concavity of a proximal concave mesh with a distal-facing concavity made primarily or entirely from metal.

18. The device in claim 1 wherein the flexible net or mesh is made by 3D printing.

19. The device in claim 1 wherein the flexible net or mesh is made by 3D printing with a flexible polymer, wherein the proximal portion of the net or mesh is thicker than the distal portion of the net or mesh.

20. The device in claim 1 wherein the flexible net or mesh is made by 3D printing with a flexible polymer, wherein the proximal portion of the net or mesh is printed with a stiffer polymer than the distal portion of the net or mesh.
